(19) United States
(12) Patent Application Publication
(10) Pub. No.: US 2010/0022437 A1
(43) Pub. Date: Jan. 28, 2010

(54) Liquid Support

(75) Inventors: Rene-Andres Artiga-Gonzalez, Duesseldorf (DE); Hubert Harth, Hilden (DE); Stefan Hammelstein, Duesseldorf (DE); Konstanze Mayer, Duesseldorf (DE); Mario Sturm, Leverkusen (DE); Karl-Heinz Scheffler, Duesseldorf (DE)

Correspondence Address: Ratner Prestia, P.O. Box 980, Valley Forge, PA, 19482, US

(73) Assignee: Henkel AG & Co. KGaA, Duesseldorf (DE)

(21) Appl. No.: 12/360,519

(22) Filed: Jan. 27, 2009

Related U.S. Application Data
(63) Continuation of application No. PCT/EP2007/055894, filed on Jun. 14, 2007.

(30) Foreign Application Priority Data
Jul. 28, 2006 (DE) 10 2006 035 746.9

(51) Int. Cl. A61K 8/11; B32B 5/16; C11D 17/00; A61K 9/50; C09D 105/00

(52) U.S. Cl. 512/4; 428/402.2; 510/444; 424/401; 424/490; 106/162.1

(57) ABSTRACT

Agglomerating carrier materials in the presence of a granulation liquid with a carbohydrate content and subsequent incorporation of a liquid, such as a perfume oil, yields fluid-containing particles that, even at an elevated liquid loading, exhibit very good powder properties and reliably retain the incorporated liquid even in aggressive solid matrices. These fluid-containing particles may be used, for example, for fragrancing purposes.

### CROSS-REFERENCE TO RELATED APPLICATIONS

This application is a continuation under 35 U.S.C. §§120 and 365(c) of International Application PCT/EP2007/055894, filed on Jun. 14, 2007. This application also claims priority under 35 U.S.C. §119 of DE 10 2006 035 746.9 filed on Jul. 28, 2006. The disclosures of PCT/EP2007/055894 and DE 10 2006 035 746.9 are incorporated herein by reference in their entirety.

### BACKGROUND OF THE INVENTION

The invention relates to fluid-containing particles which may be obtained by agglomerating carrier material in the presence of a granulation liquid comprising carbohydrates and/or the derivatives thereof, followed by exposure to a liquid to be incorporated. The invention further relates to detergent or cleaning agents which comprise such fluid-containing particles and to use of the fluid-containing particles for various fragrancing purposes.

There is a need in numerous applications for particulate carriers which are capable of absorbing liquids and, depending on the application, also storing them and releasing them again when required.

For instance, liquid constituents such as for example perfume oils or nonionic surfactants are incorporated into solid detergents or cleaning agents.

The problem with such incorporation is that the liquid can escape again when not desired, so easily giving rise to product caking. Moreover, only small quantities of liquid can often be incorporated into solid agents, such that for example the solid matrix can only absorb at most 5 wt. % or less of liquid. Furthermore, the mechanical stability of the solid matrix often suffers when it is loaded with liquid, i.e. the resultant particles become more fragile. This is undesirable because such agents are often stored in large containers, such as for example hoppers. If the particles are excessively fragile, they can easily be crushed under the pressure of their own weight.

### DETAILED DESCRIPTION OF THE INVENTION

There is accordingly furthermore a requirement for systems which are preferably capable of absorbing even a large quantity of liquids, storing them reliably and only releasing them again after some time. The object of the present invention was to meet such requirements.

This object is achieved by the subject matter of the invention which involves fluid-containing particles obtainable by agglomerating carrier material in the presence of a granulation liquid comprising carbohydrates and/or the derivatives thereof, followed by exposure to a liquid to be incorporated.

Advantageously, these particles may contain relatively large quantities of liquid, such as for example perfume oil, while nevertheless exhibiting very good flowability and mechanical stability and no tendency to form lumps. Furthermore, the liquid incorporated into the particles is now protected and may straightforwardly be incorporated into other, for example aggressive, solid matrices without there being any need to anticipate decomposition of the incorporated liquid. Taking perfume oils as an example of an incorporated liquid, this means that improved fragrance properties and improved fragrance stability are achieved.

If the liquid to be incorporated comprises
a) odoriferous substances (perfume oils)
b) liquid detergent and cleaning agent ingredients, such as preferably surfactants, in particular nonionic surfactants, silicone oils, paraffins,
c) liquid cosmetic ingredients, such as preferably oils
d) liquid non-pharmaceutical additives or active ingredients and/or
e) mixtures of the above,
   this amounts to a preferred embodiment. The use of odoriferous substances is particularly advantageous. The particles loaded with odoriferous substance are distinguished by good fragrance stability, even when they are incorporated into aggressive matrices, for example into detergent powders, in order to fragrance them.

Even if very large quantities of liquid, in particular odoriferous substances are incorporated into the particle, the particles retain their advantageous properties. According to a preferred embodiment, the particles according to the invention may contain ≧5 wt. %, preferably ≧10 wt. %, advantageously ≧15wt. %, in particular ≧20wt. % or even ≧25wt. % of incorporated liquid, preferably odoriferous substances (perfume oils).

The liquid incorporated or to be incorporated into the agglomerate may also contain solids. If the solids content of the liquid to be incorporated amounts to less than 50%, preferably less than 30%, advantageously less than 25%, in particular less than 15%, extremely preferably less than 10%, relative to the liquid to be incorporated, this amounts to a further preferred embodiment. In particular, the liquid to be incorporated contains no solids.

Perfume oils which may for example be used are individual odoriferous compounds, for example synthetic products of the ester, ether, aldehyde, ketone, alcohol and hydrocarbon type. Odoriferous substance compounds of the ester type are for example benzyl acetate, phenoxyethyl isobutyrate, p-tert.-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. Ethers include, for example, benzyl ethyl ether, the aldehydes for example include linear alkanals with 8-18 C atoms, citral, citronellal, citronellyloxyacetaldehyde, cyclamen aldehyde, hydroxycitronellal, lilial and bourgeonal, the ketones include, for example, ionones, isomethylionone and methyl cedryl ketone, the alcohols include anethole, citronellol, eugenol, geraniol, linalool, phenylethyl alcohol and terpineol, the hydrocarbons mainly include terpenes and balsams. Preferably, however, mixtures of various odoriferous substances which together produce an attractive fragrance note are used.

The perfume oils may, of course, also contain natural odoriferous substance mixtures, as are obtainable from plant or animal sources, for example stone pine, citrus, jasmine, lily, rose or ylang-ylang oil. Relatively low volatility essential oils, which are generally used as aroma components, are also suitable as perfume oils, for example sage oil, camomile oil, clove oil, melissa oil, mint oil, cinnamon leaf oil, lime blossom oil, juniper berry oil, vetiver oil, galbanum oil and labdanum oil.

According to the invention, odoriferous substances may in particular be used which are selected from odoriferous substances having
(a) an almond-like odor, such as preferably benzaldehyde, pentanal, heptenal, 5-methylfurfural, methylbutanal, furfural and/or acetophenone or
(b) an apple-like odor, such as preferably (S)-(+)-ethyl-2-methylbutanoate, diethyl malonate, ethyl butyrate, geranyl butyrate, geranyl isopentanoate, isobutyl acetate, linalyl isopentanoate, (E)-β-damascone, heptyl-2-methyl butyrate, methyl 3-methylbutanoate, 2-hexenalpentylmethyl butyrate, ethylmethyl butyrate and/or methyl 2-methylbutanoate or
(c) an apple peel-like odor, such as preferably ethyl hexanoate, hexyl butanoate and/or hexyl hexanoate or
(d) an apricot-like odor such as preferably y-undecalactone or
(e) a banana-like odor, such as preferably isobutyl acetate, isoamyl acetate, hexenyl acetate and/or pentyl butanoate or
(f) a bitter almond-like odor such as preferably 4-acetyltoluene or
(g) a blackcurrant-like odor such as preferably mercaptomethylpentanone and/or methoxymethylbutanethiol or
(h) a citrus-like odor such as preferably linalyl pentanoate, heptanal, linalyl isopentanoate, dodecanal, linalyl formate, α-p-dimethylstyrene, p-cymenol, nonanal, β-cubebene, (Z)-limonene oxide, cis-6-ethenyltetrahydro-2,2,6-trimethylpyran-3-ol, cis-pyranoid linalool oxide, dihydrolinalool, 6(10)-dihydromyrcenol, dihydromyrcenol, β-farnesene, (Z)-β-farnesene, (Z)-ocimene, (E)-limonene oxide, dihydroterpinyl acetate, (+)-limonene, (epoxymethylbutyl)-methylfuran and/or p-cymene or
(i) a cocoa-like odor such as preferably dimethylpyrazine, butylmethyl butyrate and/or methylbutanal or
(j) a coconut-like odor, such as preferably γ-octalactone, γ-nonalactone, methyl laurate, tetradecanol, methyl nonanoate, (3S,3aS,7aR)-3a,4,5,7a-tetrahydro-3, 6-dimethylbenzofuran-2(3H)-one, 5-butyldihydro-4-methyl-2(3H)-furanone, ethyl undecanoate and/or 6-decalactone or
(k) a cream-like odor such as preferably diethyl acetal, 3-hydroxy-2-butanone, 2,3-pentadione and/or 4-heptenal or
(l) a flower-like odor such as preferably benzyl alcohol, phenylacetic acid, tridecanal, p-anisyl alcohol, hexanol, (E,E)-farnesyl acetone, methyl geranate, trans-crotonaldehyde, tetradecyl aldehyde, methyl anthranilate, linalool oxide, epoxylinalool, phytol, 10-epi-γ-eudesmol, nerol oxide, ethyl dihydrocinnamate, γ-dodecalactone, hexadecanol, 4-mercapto-4-methyl-2-pentanol, (Z)-ocimene, cetyl alcohol, nerolidol, ethyl (E)-cinnamate, elemicin, pinocarveol, α-bisabolol, (2R,4R)-tetrahydro-4-methyl-2-(2-methyl-1-propenyl)-2H-pyran, (E)-isoelemicin, methyl 2-methylpropanoate, trimethylphenylbutenone, 2-methylanisole, β-farnesol, (E)-isoeugenol, nitrophenylethane, ethyl vanillate, 6-methoxyeugenol, linalool, β-ionone, trimethylphenylbutenone, ethyl benzoate, phenylethyl benzoate, isoeugenol and/or acetophenone or
(m) a freshness odor such as preferably methyl hexanoate, undecanone, (Z)-limonene oxide, benzyl acetate, ethyl hydroxyhexanoate, isopropyl hexanoate, pentadecanal, β-elemene, α-zingiberene, (E)-limonene oxide, (E)-p-mentha-2,8-dien-1-ol, menthone, piperitone, (E)-3-hexenol and/or carveol or
(n) a fruit odor such as preferably ethylphenyl acetate, geranyl valerate, γ-heptalactone, ethyl propionate, diethyl acetal, geranyl butyrate, ethyl heptylate, ethyl octanoate, methyl hexanoate, dimethylheptenal, pentanone, ethyl 3-methylbutanoate, geranyl isovalerate, isobutyl acetate, ethoxypropanol, methyl-2-butenal, methyinonanedione, linalyl acetate, methyl geranate, limonene oxide, hydrocinnamic alcohol, diethyl succinate, ethyl hexanoate, ethyl methylpyrazine, neryl acetate, citronellyl butyrate, hexyl acetate, nonyl acetate, butylmethyl butyrate, pentenal, isopentyldimethylpyrazine, p-menth-1-en-9-ol, hexadecanone, octyl acetate, γ-dodecalactone, epoxy-β-ionone, ethyl octenoate, ethyl isohexanoate, isobornyl propionate, cedrenol, p-menth-1-en-9-yl acetate, cadinadiene, (Z)-3-hexenyl hexanoate, ethyl cyclohexanoate, 4-methylthio-2-butanone, 3,5-octadienone, methylcyclohexane carboxylate, 2-pentylthiophene, α-ocimene, butanediol, ethyl valerate, pentanol, isopiperitone, butyl octanoate, ethyl vanillate, methyl butanoate, 2-methylbutyl acetate, propyl hexanoate, butyl hexanoate, isopropyl butanoate, spathulenol, butanol, δ-dodecalactone, methylquinoxaline, sesquiphellandrene, 2-hexenol, ethyl benzoate, isopropyl benzoate, ethyl lactate and/or citronellyl isobutyrate or
(o) a geranium-like odor, such as preferably geraniol, (E,Z)-2,4-nonadienal, octadienone and/or o-xylene or
(p) a grape-like odor such as preferably ethyl decanoate and/or hexanone or
(q) a grapefruit-like odor such as preferably (+)-5,6-dimethyl-8-isopropenylbicyclo[4.4.0]dec-1-en-3-one and/or p-menthenethiol or
(r) a grass-like odor such as preferably 2-ethylpyridine, 2,6-dimethylnaphthalene, hexanal and/or (Z)-3-hexenol or
(s) a green note, preferably 2-ethylhexanol, 6-decenal, dimethylheptenal, hexanol, heptanol, methyl-2-butenal, hexyl octanoate, nonanoic acid, undecanone, methyl geranate, isobornyl formate, butanal, octanal, nonanal, epoxy-2-decenal, cis-linalool, pyran oxide, nonanol, alpha,γ-dimethylallyl alcohol, (Z)-2-penten-1-ol, (Z)-3-hexenyl butanoate, isobutylthiazole, (E)-2-nonenal, 2-dodecenal, (Z)-4-decenal, 2-octenal, 2-hepten-1-al, bicyclogermacrene, 2-octenal, α-thujene, (Z)-β-farnesene, (−)-γ-elemene, 2,4-octadienal, fucoserratene, hexenyl acetate, geranyl acetone, valencene, β-eudesmol, 1-hexenol, (E)-2-undecenal, Artemisia ketone, viridiflorol, 2,6-nonadienal, trimethylphenylbutenone, 2,4-nonadienal, butyl isothiocyanate, 2-pentanol, elemol, 2-hexenal, 3-hexenal, (+)-(E)-limonene oxide, cis-isocitral, dimethyloctadienal, bornyl formate, bornyl isovalerate, isobutyraldehyde, 2,4-hexadienal, trimethylphenylbutenone, nonanone, (E)-2-hexenal, (+)-cis-rose oxide, menthone, coumarin, (epoxymethylbutyl)-methylfuran, 2-hexenol, (E)-2-hexenol and/or carvyl acetate or
(t) a green tea-like odor, preferably (−)-cubenol or
(u) a herb-like odor, preferably octanone, hexyl octanoate, caryophyllene oxide, methylbutenol, safranal, benzyl benzoate, bornyl butyrate, hexyl acetate, β-bisabolol, piperitol, β-selinene, α-cubebene, p-menth-1-en-9-ol, 1,5,9,9-tetramethyl-12-oxabicyclododeca-4,7-diene, T-muurolol, (−)-cubenol, levomenol, ocimene, α-thujene, p-menth-1-en-9-yl acetate, dehydrocarveol, Artemisia alcohol, γ-muurolene, hydroxypentanone, (Z)-ocimene, β-elemene, δ-cadinol, (E)-β-ocimene, (Z)-dihydrocarvone, α-cadinol, calamenene, (Z)-piperitol, lavandulol, β-bourbonene, (Z)-3-hexenyl 2-methylbutanoate, 4-(1-methylethyl)-benzenemethanol, Artemisia ketone, methyl-2-butenol, heptanol, (E)-dihydrocarvone, p-2-menthen-1-ol, α-curcumene, spathulenol, sesquiphellandrene, citronellyl valerate, bornyl isovalerate, 1,5-octadien-3-ol, methyl benzoate, 2,3,4,5-tetrahydroanisole and/or hydroxycalamenene or
(v) a honey-like odor, preferably ethyl cinnamate, β-phenethyl acetate, phenylacetic acid, phenylethanal, methyl anthranilate, cinnamic acid, β-damascenone, ethyl (E)-cinnamate, 2-phenylethyl alcohol, citronellyl valerate, phenylethyl benzoate and/or eugenol or
(w) a hyacinth-like odor, preferably hotrienol or
(x) a jasmine-like odor, preferably methyl jasmonate, methyl dihydroepijasmonate and/or methyl epijasmonate or
(y) a lavender-like odor, preferably linalyl valerate and/or linalool or
(z) a lemon-like odor, preferably neral, octanal, δ-3-carene, limonene, geranial, 4-mercapto-4-methyl-2-pentanol, citral, 2,3-dehydro-1,8-cineole and/or α-terpinene or
(aa) a lily-like odor, preferably dodecanal or
(bb) a magnolia-like odor, preferably geranyl acetone, or
(cc) a mandarin-like odor, preferably undecanol or
(dd) a melon-like odor, preferably dimethylheptenal, or
(ee) a mint-like odor, preferably menthone, ethyl salicylate, p-anisaldehyde, 2,4,5, 7a-tetrahydro-3,6-dimethylbenzofu ran, epoxy-p-menthene, geranial, (methylbutenyl)-methylfuran, dihydrocarvyl acetate, β-cyclocitral, 1,8-cineole, β-phellandrene, methylpentanone, (+)-limonene, dihydrocarveol, (−)-carvone, (E)-p-mentha-2,8-dien-1-ol, isopulegyl acetate, piperitone, 2,3-dehydro-1,8-cineole, α-terpineol, DL-carvone and/or α-phellandrene or
(ff) a nut-like odor, preferably 5-methyl(E)-2-hepten-4-one, γ-heptalactone, 2-acetyl pyrrole, 3-octen-2-one, dihydromethylcyclopentapyrazine, acetylthiazole, 2-octenal, 2,4-heptadienal, 3-octenone, hydroxypentanone, octanol, dimethylpyrazine, methylquinoxaline and/or acetylpyrroline or
(gg) an orange-like odor, preferably methyl octanoate, undecanone, decyl alcohol, limonene and/or 2-decenal or
(hh) an orange peel-like odor, preferably decanal and/or β-carene or
(ii) a peach-like odor, preferably γ-nonalactone, (Z)-6-dodecene-γ-lactone, δ-decalactone, R-δ-decenolactone, hexyl hexanoate, 5-octanolide, γ-decalactone and/or δ-undecalactone or
(jj) a peppermint-like odor, preferably methyl salicylate and/or l-menthol or
(kk) a pine-like odor, preferably α-p-dimethylstyrene, β-pinene, bornyl benzoate, β-terpinene, dihydroterpinyl acetate and/or α-pinene or
(ll) a pineapple-like odor, preferably propyl butyrate, propyl propanoate and/or ethyl acetate or
(mm) a plum-like odor, preferably benzyl butanoate, or
(nn) a raspberry-like odor, preferably β-ionone or
(oo) a rose-like odor, preferably β-phenethyl acetate, 2-ethylhexanol, geranyl valerate, geranyl acetate, citronellol, geraniol, geranyl butyrate, geranyl isovalerate, citronellyl butyrate, citronellyl acetate, isogeraniol, tetrahydro-4-methyl-2-(2-methyl-1-propenyl)-2,5-cis-2H-pyran, isogeraniol, 2-phenylethyl alcohol, citronellyl valerate and/or citronellyl isobutyrate, or
(pp) a green mint-like odor, preferably carvyl acetate and/or carveol, or
(qq) a strawberry-like odor, preferably hexyl methylbutyrate, methyl cinnamate, pentenal, methyl cinnamate or
(rr) a sweetish odor, preferably benzyl alcohol, ethylphenyl acetate, tridecanal, nerol, methyl hexanoate, linalyl isovalerate, undecanaldehyde, caryophyllene oxide, linalyl acetate, safranal, uncineol, phenylethanal, p-anisaldehyde, eudesmol, ethylmethylpyrazine, citronellyl butyrate, 4-methyl-3-penten-2-one, nonyl acetate, 10-epi-γ-eudesmol, β-bisabolol, (Z)-6-dodecene-γ-lactone, β-farnesene, 2-dodecenal, γ-dodecalactone, epoxy-β-ionone, 2-undecenal, styrene glycol, methyl furaneol, (−)-cis-rose oxide, (E)-β-ocimene, dimethylmethoxyfuranone, 1,8-cineole, ethylbenzaldehyde, 2-pentylthiophene, α-farnesene, methionol, 7-methoxycoumarin, (Z)-3-hexenyl 2-methylbutanoate, o-aminoacetophenone, viridiflorol, isopiperitone, p-sinensal, ethyl vanillate, methyl butanoate, p-methoxystyrene, 6-methoxyeugenol, 4-hexanolide, ≢-dodecalactone, sesquiphellandrene, diethyl malate, linalyl butyrate, guaiacol, coumarin, methyl benzoate, isopropyl benzoate, safrole, durene, γ-butyrolactone, ethyl isobutyrate and/or furfural or
(ss) a vanilla-like odor, preferably vanillin, methyl vanillate, acetovanillone and/or ethyl vanillate, or
(tt) a water melon-like odor, preferably 2,4-nonadienal or
(uu) a wood-like odor, preferably α-muurolene, cadina-1,4-dien-3-ol, isocaryophyllene, eudesmol, α-ionone, bornyl butyrate, (E)-α-bergamotene, linalool oxide, ethylpyrazine, 10-epi-γ-eudesmol, α-ionone, bornyl butyrate, (E)-α-bergamotene, linalool oxide, ethylpyrazine, 10-epi-γ-eudesmol, germacrene B, trans-sabinene hydrate, dihydrolinalool, isodihydrocarveol, β-farnesene, β-sesquiphellandrene, α-elemene, α-calacorene, epoxy-β-ionone, germacrene D, bicyclogermacrene, alloaromadendrene, α-thujene, oxo-β-ionone, (−)-γ-elemene, γ-muurolene, sabinene, α-guaiene, α-copaene, γ-cadinene, nerolidol, β-eudesmol, α-cadinol, δ-cadinene, 4,5-dimethoxy-6-(2-propenyl)-1,3-benzodioxole, [1ar-(1a-alpha,4a-alpha,7alpha,7a-beta,7b-alpha)]-decahydro-1,1,7-trimethyl-4-methylene-1H-cycloprop[e]azulene, α-gurjunene, guaiol, α-farnesene, γ-selinene, 4-(1-methylethyl)-benzenemethanol, perillene, elemol, α-humulene, β-caryophyllene and/or β-guaiene or
(vv) mixtures of the above.

The liquid incorporated or to be incorporated into the agglomerate may preferably contain liquid cosmetic ingredients, such as for example oils. Preferred oils may advantageously include fully synthetic oils such as for example silicone oils, plant and/or animal fatty oils (triglycerides of moderately or unsaturated fatty acids) and/or essential oils (for example from plant parts).

The liquid incorporated or to be incorporated into the agglomerate may preferably contain one or more skin-conditioning and/or skin-protecting active substances. Skin-conditioning active substances are any such active substances which impart an organoleptic and/or cosmetic advantage to the skin. Skin-conditioning active substances are preferably selected from the following substances:
a) waxes such as for example carnauba, spermaceti, beeswax, lanolin and/or derivatives thereof and others.
b) hydrophobic plant extracts
c) hydrocarbons such as for example squalene and/or squalane
d) higher fatty acids, preferably those with at least 12 carbon atoms, for example lauric acid, stearic acid, behenic acid, myristic acid, palmitic acid, oleic acid, linoleic acid, linolenic acid, isostearic acid and/or polyunsaturated fatty acids and others.
e) higher fatty alcohols, preferably those having at least 12 carbon atoms, for example lauryl alcohol, cetyl alcohol, stearyl alcohol, oleyl alcohol, behenyl alcohol, cholesterol and/or 2-hexadecanol and others.
f) esters, preferably those such as cetyl octanoate, lauryl lactate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactates, alkyl citrates and/or alkyl tartrates and others.
g) lipids such as for example cholesterol, ceramides and/or sucrose esters and others.
h) vitamins such as for example vitamins A and E, vitamin alkyl esters, including vitamin C alkyl esters and others.
i) sunscreen preparation
j) phospholipids
k) derivatives of alpha-hydroxy acids
l) odoriferous substances
m) germicides for cosmetic use, both synthetic, such as for example salicylic acid and/or others, and natural, such as for example neem oil and/or others.
n) silicones
and mixtures of any of the above-stated components.

The liquid incorporated or to be incorporated into the agglomerate may preferably contain antiseptically active oil, preferably an essential oil, which is in particular selected from the group of garden angelica—Angelica archangelica, aniseed—Pimpinella anisum, Siam benzoin—Styrax tokinensis, cabreuva—Myrocarpus fastigiatus, cajeput—Melaleuca leucadendron, rock rose—Cistrus ladaniferus, copaiba balsam—Copaifera reticulata, costus root—Saussurea discolor, silver fir needle—Abies alba, elemi—Canarium luzonicum, fennel—Foeniculum dulce, pine needle—Picea abies, geranium—Pelargonium graveolens, ho leaves—Cinnamonum camphora, immortelle (everlasting flower)—Helichrysum ang., ginger, extra—Zingiber off., St. John's wort—Hypericum perforatum, jojoba, German chamomile—Matricaria recutita, blue chamomile—Matricaria chamomilla, Roman chamomile—Anthemis nobilis, wild chamomile—Ormensis multicaulis, carrot—Daucus carota, mountain pine—Pinus mugho, lavandin—Lavandula hybrida, Litsea cubeba—May Chang manuka—Leptospermum scoparium, melissa—Melissa officinalis, maritime pine—Pinus pinaster, myrrh—Commiphora molmol, myrtle—Myrtus communis, neem—Azadirachta, niaouli—(MQV) Melaleuca quin. viridiflora, palmarosa—Cymbopogon martini, patchouli—Pogostemon patchouli, Peru balsam—Myroxylon balsamum var. pereirae, Ravensara aromatica, rosewood—Aniba rosaeodora, sage—Salvia officinalis, horsetail—Equisetaceae, yarrow, extra—Achillea millefolia, ribwort—Plantago lanceolata, styrax—Liquidambar orientalis, tagetes (marigold)—Tagetes patula, tea tree—Melaleuca alternifolia, Tolu balsam—Myroxylon balsamum L., Virginia cedar—Juniperus virginiana, frankincense (olibanum)—Boswellia carteri, white fir—Abies alba.

The liquid incorporated or to be incorporated into the agglomerate may preferably contain skin-protecting active substances, advantageously skin-protecting oil. The skin-protecting substance advantageously comprises a skin-protecting oil, for example also a carrier oil, in particular selected from the group algae oil—Phaeophyceae oil, Aloe vera oil—Aloe vera brasiliana, apricot kernel oil—Prunus armeniaca, arnica oil—Arnica montana, avocado oil—Persea americana, borage oil—Borago officinalis, calendula oil—Calendula officinalis, camellia oil—Camellia oleifera, thistle oil—Carthamus tinctorius, peanut oil—Arachis hypogaea, hemp oil—Cannabis sativa, hazelnut oil—Corylus avellana, St. John's wort oil—Hypericum perforatum, jojoba oil—Simmondsia chinensis, carrot oil—Daucus carota, coconut oil—Cocos nucifera, pumpkin seed oil—Curcubita pepo, candlenut oil—Aleurites moluccana, macadamia nut oil—Macadamia ternifolia, almond oil—Prunus dulcis, olive oil—Olea europaea, peach stone oil—Prunus persica, rapeseed oil—Brassica oleifera, castor oil—Ricinus communis, black cumin oil—Nigella sativa, sesame oil—Sesamium indicum, sunflower oil—Helianthus annus, grapeseed oil—Vitis vinifera, walnut oil—Juglans regia, wheat germ oil—Triticum sativum, with borage oil, hemp oil and almond oil being particularly advantageous among these.

The liquid incorporated or to be incorporated into the agglomerate may preferably contain humectant factors, for example those selected from the following group: amino acids, chitosan or chitosan salts/derivatives, ethylene glycol, glucosamine, glycerol, diglycerol, triglycerol, uric acid, honey and hardened honey, creatinine, breakdown products of collagen, lactitol, polyols and polyol derivatives (for example butylene glycol, erythritol, propylene glycol, 1,2,6-hexanetriol, polyethylene glycols, such as PEG-4, PEG-6, PEG-7, PEG-8, PEG-9, PEG-10, PEG-12, PEG-14, PEG-16, PEG-18, PEG-20), pyrrolidonecarboxylic acid, sugars and sugar derivatives (for example fructose, glucose, maltose, maltitol, mannitol, inositol, sorbitol, sorbityl silanediol, sucrose, trehalose, xylose, xylitol, glucuronic acid and the salts thereof), ethoxylated sorbitol (sorbeth-6, sorbeth-20, sorbeth-30, sorbeth-40), hardened starch hydrolysates and mixtures of hardened wheat protein and PEG-20 acetate copolymer, in particular panthenol.

Any known carrier materials may in principle be considered as carriers. However, if the carrier material comprises an anionic carrier material, advantageously zeolite and/or phyllosilicate, preferably bentonite, this amounts to a preferred embodiment.

The presence of an inorganic excipient also provides optical advantages, because the resultant particles may consequently take on a crystalline appearance, in particular if solid added materials in crystal form, preferably sugar and/or salt (for example common salt, sea salt, etc.) are, for example, added before agglomeration.

Accordingly, if solid added materials in crystal form, preferably sugar and/or salt, are added to the carrier material before agglomeration, advantageously in quantities of ≧10 wt. %, more advantageously of ≧20 wt. %, in further advantageous manner ≧30 wt. %, in particular in quantities of 40-55 wt. % relative to the entire solids to be agglomerated, this amounts to a preferred embodiment.

Fluid-containing particles according to such an embodiment are distinguished in that they exhibit a particularly attractive crystal form and are nevertheless capable of containing large quantities of liquid. The crystal form gives the consumer the impression of a visually high-quality product.

Alternatively, the optional addition of sugar and/or salt may also be made just before exposure to the liquid to be incorporated. Accordingly, if solid added materials in crystal form, preferably sugar and/or salt, are added to the carrier material after agglomeration, advantageously in quantities of ≧10 wt. %, more advantageously of ≧20 wt. %, in further advantageous manner ≧30 wt. %, in particular in quantities of 40-55 wt. % relative to the entire solids to be agglomerated, this amounts to a preferred embodiment. In such a case, the agglomerated carrier material and the sugar and/or the salt are thus blended (mixed) with one another before exposure to the liquid to be incorporated.

For the purposes of the invention, it is generally advantageous, independently of the addition of solid added materials in crystal form, for the fluid-containing particles to contain ≧30 wt. %, preferably ≧40 wt. %, advantageously ≧50 wt. %, in particular ≧60 wt. % of zeolite and/or bentonite.

If the carrier material to be used according to the invention comprises spray-dried material, preferably tower powder, this amounts to a particularly preferred embodiment.

The tower powder is obtained in known manner by spray drying. The material to be dried (liquid solution or suspension, for example an aqueous suspension of thermally stable detergent ingredients which neither volatilize nor break down under the conditions of spray drying, such as for example surfactants, builders, fillers) is broken down into a fine mist at the top of a wide, cylindrical container by nozzles or by means of a rapidly rotating atomizing disk. For example hot air (for example with a temperature of 250 to 350° C.) or also an inert gas are passed from below countercurrently to the resultant spray cone. The drying gas may also be supplied cocurrently from above, for example in the case of highly thermally sensitive products (for example enzymes, active microorganisms). The dried material falls downwards as a powder of greater or lesser fineness, as a granular product or in the form of small beads (prills) and is discharged at the bottom of the dryer.

According to a preferred embodiment of the present invention, the fluid-containing particles according to the invention are distinguished in that the carrier material has been agglomerated at elevated pressure (preferably >10000 Pa). Such an agglomeration process in particular preferably proceeds under elevated pressure by means of rollers, with the slugs subsequently being broken up. Pressures in excess of 10000 Pa are preferably required for this type of agglomeration.

According to the invention, the granulation liquid comprises carbohydrates and/or the derivatives thereof; modified carbohydrates, such as for example carboxymethylcellulose (CMC), are thus also included. The granulation liquid is preferably aqueous, advantageously with a CMC content ≦8 wt. %, advantageously of <6 wt. %, in particular of 5.5 wt. % to 3 wt. %.

If the granulation liquid according to the invention contains
(a) cellulose (derivatives) such as carboxymethylcellulose, carboxymethylhydroxyethylcellulose, hydroxypropylmethylcellulose, methylcellulose, ethylcellulose, methylhydroxyalkylcellulose, hydroxyethylcellulose, hydroxypropylcellulose, ethylhydroxyethylcellulose, cationically modified hydroxyethylcellulose and/or hydroxypropylcellulose,
(b) starch (derivatives) such as carboxymethyl starch (starch carboxymethyl ether), hydroxyethyl starches, hydroxypropyl starches, cationic starch (derivatives)
(c) mono-, oligo- and/or polysaccharide(s) (derivative(s)), such as in particular ribose, xylose and arabinose, glucose, mannose, galactose, fructose, sorbose, fucose, rhamnose, sucrose (cane sugar or beet sugar), maltose (malt sugar), lactose (milk sugar), trehalose, cellobiose, gentiobiose, isomaltose, lactulose, melibiose, neohesperidose, neotrehalose, nigerose, palatinose, xanthan, dextran, chitosan and/or alginates
(d) mixtures of the above,
this likewise amounts to a preferred embodiment.

The agglomeration according to the invention is advantageously distinguished in that the particles grow (preferably to sizes of 0.2 to 1.4 mm) during the process in such a manner that the agglomerated particles are not sealed against the uptake of liquids. Aqueous solutions of carboxymethylcelluloses are extremely highly preferred granulation liquids for this purpose.

If the granulation liquid according to the invention has a solvent content, preferably water content, of greater than 80 wt. %, advantageously of 90-99 wt. %, advantageously of 94-98 wt. %, this amounts to a further preferred embodiment.

If the granulation liquid according to the invention has a content of carbohydrates and/or derivatives of ≦10 wt. %, advantageously of ≦8 wt. %, advantageously of ≦6 wt. %, in particular of 5.5 wt. % to 3 wt. %, this amounts to a further preferred embodiment.

Granules according to the invention may accordingly be produced by intensively mixing spray-dried zeolite powder for example in a ploughshare mixer with an aqueous CMC solution and then removing the water in a fluidized bed. The resultant granules may thereafter be impregnated with the liquid components, such as for example perfume. According to a preferred embodiment, the water is thus at least in part removed from the granules before exposure to the liquid components, such as for example perfume. In such a case, the water content is preferably adjusted to <25 wt. %, preferably <20 wt. %, advantageously <15 wt. %, more advantageously <10 wt. %, in particular <6 wt. % or even <4 wt. %, for example by treatment in a fluidized bed. Water contents of at most 3 wt. % or at most 2 wt. % or at most 1 wt. % are also possible. Any water adhering to optionally present aluminosilicates such as zeolites was in each case not included in these calculations.

By agglomerating carrier material in the presence of a granulation liquid with a carbohydrate content, optionally followed by subsequent reduction in water content, and subsequent incorporation of a liquid, such as for example a perfume oil, fluid-containing particles are thus obtained which, even at an elevated liquid loading, exhibit very good powder properties and reliably retain the incorporated liquid even in the presence of aggressive solid matrices. These fluid-containing particles may be used, for example, for many and varied fragrancing purposes.

As has already be explained, the fluid-containing particles according to the invention are capable of containing relatively large quantities of liquid. They thus act as a kind of liquid depot, as the contained liquid, for example perfume, can be released again on use, for example during laundering, and exert its action, for example fragrancing in the case of perfume. However, before being used, the stored liquid remains reliably incorporated in the particles.

It is also possible to incorporate ingredients into the particles which are solid at room temperature, but are liquid at elevated temperatures, preferably such ingredients having melting points in the temperature range from 30° C. to 260° C., such as for example polyesters or carbohydrates such as for example disaccharides from the isomaltitol family.

The fluid-containing particles may be coated, for example in order further to improve this storage action of the particles according to the invention or alternatively also independently thereof. Coated fluid-containing particles, which are preferably powder coated and/or film coated, constitute a preferred embodiment of the invention.

The coating according to the invention may comprise colored substances, dyes, brighteners and/or pigments, advantageously in the nanoscale range or in the micrometer range, this being a preferred embodiment.

Fluid-containing particles according to the invention which are coated with a thermoplastic material, such as preferably PEG, PVA, polyacrylates, PVP, carbohydrates, polyesters such as preferably PET, constitute a preferred embodiment of the invention.

With regard to a possibly desired crystal-like appearance of the particles according to the invention, the use of the above-stated thermoplastics such as for example PEG with average molecular weights of between 3000 and 10000 is very advantageous, since these thermoplastics may form crystal-like shapes on solidifying.

Any other coating compositions may also be used for the coating according to the invention. Coating compositions are for example substances which impart for example a glossy appearance to the outer surface of the object (to be coated) and/or form a coating (a shell) on the outer surface. Solid and/or liquid substances may be used as coating compositions, preferably those capable of suppressing or delaying moisture penetration or preventing or delaying aroma losses.

Suitable coating compositions may for example contain water-soluble, water-dispersible and/or water-insoluble (co)polymers. The coating layer itself may be for example water-soluble or water-insoluble.

Water-soluble polymers contain a number of hydrophilic groups which are sufficient for water solubility and are advantageously not crosslinked. The hydrophilic groups may be nonionic, anionic, cationic or zwitterionic, for example —NH₂, —OH, —SH, —O—, —COOH, —COO⁻M⁺, —SO₃⁻M⁺, —PO₃²⁻M²⁺, —NH₃₊,

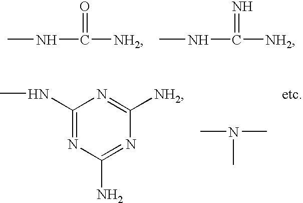

The individual polymers may simultaneously contain different hydrophilic groups, for example ionic and nonionic and/or anionic together with cationic groups. Preferred water-soluble polymers may be for example natural polysaccharides and/or polypeptides, such as for example starch, alginates, pectins, vegetable gums, casein, gelatin etc. Preferred water-soluble polymers may for example be semi-synthetic polymers, such as for example cellulose ethers or starch ethers. Preferred water-soluble polymers may be for example biotechnologically manufactured products, such as for example pullulan, curdlan or xanthan. Preferred water-soluble polymers may inter alia be for example synthetic polymers, such as for example homo- and/or copolymers of (meth)acrylic acid and the derivatives thereof, of maleic, vinylsulfonic, vinylphosphonic acid, polyvinyl alcohol, polyethyleneimine, polyvinylpyrrolidone. Preferred coating compositions contain a water-soluble (co)polymer, in particular with a melting or softening point in the range from 48° C. to 300° C., advantageously in the range from 48° C. to 200° C., more advantageously in the range from 48° C. to 200° C. Suitable water-soluble (co)polymers with a corresponding melting or softening point may advantageously be selected from the group consisting of polyalkylene glycols, polyethylene terephthalate, polyvinyl alcohols and mixtures thereof.

In addition to the actual coating composition or also independently thereof, the optional coating may comprise further constituents, such as for example advantageously textile softening compounds and/or perfume.

It is also possible to coat the particles repeatedly, for example by first enclosing the particles in a first coating, for example containing a textile softening compound, and subsequently providing the resultant object with a further shell, for example containing water-soluble polymer and perfume.

According to a preferred embodiment, the optional coating contains lipids and/or silicone oils. Preferred lipids are
(a) lipophilic hydrocarbons (such as for example also triacontane, squalene or carotenoids, etc.),
(b) lipophilic alcohols (such as for example wax alcohols, retinol or cholesterol etc.),
(c) ether lipids
(d) lipophilic carboxylic acids (fatty acids),
(e) lipophilic esters [such as neutral fats, i.e. mono-, di- and triacylglycerins (triglycerides), sterol esters etc.]
(f) lipophilic amides (such as for example ceramides etc.),
(g) waxes
(h) lipids with more than 2 hydrolysis products, such as for example glycolipids, phospholipids, sphingolipids and/or glycerolipids etc.
(i) lipids in the form of relatively high molecular weight conjugates with more than 2 hydrolysis products, such as for example lipoproteins and/or lipopolysaccharides etc.,
(j) phosphorus-free glycolipids, such as for example glycosphingolipids (such as preferably cerebrosides, gangliosides, sulfatides) or such as for example glycoglycerolipids (such as preferably glycosyl di- and monoglycerides) etc.
(k) carbohydrate-free phospholipids, such as for example sphingophospholipids (such as preferably sphingomyelins) or such as for example glycerophospholipids (such as preferably lecithins, cephalins, cardiolipins, phosphatidylinositols and phosphatidylinositol phosphates etc.)
(l) mixtures of the above.

In a further preferred embodiment, the optional coating comprises colored substances or dyes, brighteners and/or pigments, advantageously in the nanoscale range or in the micrometer range, preferably white pigments, in particular selected from titanium dioxide pigments, such as in particular anatase pigments and/or rutile pigments, zinc sulfide pigments, zinc oxide (zinc white), antimony trioxide (antimony white), basic lead carbonate (lead white) 2PbCO₃.Pb(OH)₂, lithopone ZnS+BaSO₄. White auxiliary substances, such as preferably calcium carbonate, talcum 3MgO.4SiO₂.H₂O and/or barium sulfate may preferably also be present.

In a further preferred embodiment, the pigments which may preferably be constituents of an optional coating comprise
(a) colored pigments (preferably inorganic colored pigments, in particular iron oxide pigments, chromate pigments, iron blue pigments, chromium oxide pigments, ultramarine pigments, oxide mixed phase pigments and/or bismuth vanadate pigments),
(b) black pigments (for example aniline black, perylene black, iron oxide pigments, manganese black and/or spinell black),
(c) lustrous pigments (preferably special effect pigments in flake form, metal special effect pigments such as for example aluminum pigments (silver bronze), copper pigments and copper/zinc pigments (gold bronzes) and zinc pigments, pearlescent pigments, such as for example magnesium stearate, zinc stearate, lithium stearate or ethylene glycol distearate or polyethylene terephthalate, interference pigments such as for example metal oxide/mica pigments) and/or
(d) luminescent pigments such as for example fluorescent azomethine yellow, silver-doped and/or copper-doped zinc sulfide pigments.

The optional coating may preferably also comprise the following substances:
(a) carbonates, such as preferably chalk, limestone powder, calcite and/or precipitated calcium carbonate, dolomite and/or barium carbonate
(b) sulfates, such as preferably barytes, blanc fixe and/or calcium sulfate
(c) silicates, such as preferably talcum, pyrophyllite, chlorite, hornblende, mica, kaolin
(a) wollastonite, slate flour, precipitated Ca, Al, Ca/Al, Na/Al silicates, feldspars and/or mullite
(d) silicas, such as preferably quartz, fused silica, cristobalite, diatomaceous earth, Neuburg siliceous earth, precipitated silica, pyrogenic silica, powdered glass, powdered pumice, perlite, Ca metasilicates and/or fibers prepared from melts of glass, basalts, slags
(e) oxides, such as in particular aluminum hydroxide and/or magnesium hydroxide
(f) organic fibers, such as in particular textile fibers, cellulose fibers, polyethylene fibers, polypropylene fibers, polyamide fibers, polyacrylonitrile fibers and/or polyester fibers, preferably with lengths in the nanometer or micrometer range and/or
(g) flours, such as for example starch flours.

Using such pigments may further enhance a desired crystalline appearance of the particles.

According to a further preferred embodiment, the optional coating of the particles according to the invention is pH- and/or temperature- and/or ionic strength-sensitive or contains pH- and/or temperature- and/or ionic strength-sensitive materials.

The terms pH-sensitivity, temperature-sensitivity and/or ionic strength-sensitivity are here taken to mean that, in the event of a change in pH value, temperature and/or ionic strength in the medium to which the coating is exposed (for example a washing liquor), the coating or the materials forming the coating materials undergo
(a) a change (increase or decrease) in solubility (preferably in water); and/or
(b) a change (increase or decrease) in diffusion density; and/or
(c) a change (acceleration or retardation) of dissolution kinetics; and/or
(d) a change (increase or decrease) in mechanical stability.

For temperature-sensitivity, in addition to the stated options (a) to (d), there is also the additional option (e), according to which, in the event of a change in temperature, the coating or the materials forming the coating undergo a change in state of matter from solid to liquid or vice versa, i.e. the materials melt or solidify.

If the average size of the particles according to the invention is between 0.1 and 2.0 mm, preferably 0.15 and 1.5 mm, in particular 0.2 and 1.2 mm, this is a preferred embodiment of the invention.

A preferred embodiment of the invention is also obtained if the fluid-containing particles contain solids from the group of silicates, phosphates, urea and/or the derivatives thereof, sulfates, carbonates, citrates, citric acid, acetates and/or salts of anionic surfactants.

Preferably, however, a particle according to the invention contains solids from the group of silicates, phosphates, urea and/or the derivatives thereof, sulfates, carbonates, citrates, citric acid, acetates and/or salts of anionic surfactants in only a limited quantity, for example in quantities of ≦60 wt. %, ≦50 wt. %, ≦40 wt. %, ≦30 wt. %, ≦20 wt. %, ≦10 wt. % or ≦5 wt. %, relative to the entire particles. According to a preferred embodiment, the particles according to the invention may contain absolutely no solids from the group of silicates, phosphates, urea and/or the derivatives thereof, sulfates, carbonates, citrates, citric acid, acetates and/or salts of anionic surfactants.

In general, however, it may be advantageous for the fluid-containing particles according to the invention to comprise further ingredients. If they contain at least one further, preferably two further or more substances conventionally present in detergents or cleaning agents, preferably a substance from the group of surfactants, builder substances (inorganic and organic builder substances), bleaching agents, bleaching activators, bleaching stabilizers, bleaching catalysts, enzymes, specific polymers (for example those having cobuilder properties), graying inhibitors, optical brighteners, UV protective substances, soil repellents, electrolytes, colorants, odoriferous substances, fragrances, perfume carriers, pH adjusting agents, complexing agents, fluorescent agents, foam inhibitors, anti-crease agents, antioxidants, quaternary ammonium compounds, antistatic agents, ironing aids, UV absorbers, antiredeposition agents, germicides, antimicrobial active ingredients, fungicides, viscosity regulators, pearlescent agents, dye transfer inhibitors, shrinkage prevention agents, corrosion inhibitors, preservatives, softeners, rinse conditioners, protein hydrolysates, waterproofing and impregnation agents, hydrotropes, silicone oils and anti-swelling and anti-slip agents, this amounts to a preferred embodiment.

It is also possible to incorporate ingredients into the particle which are solid at room temperature (20° C.), but are liquid at elevated temperatures, advantageously selected from the group of fatty alcohols, fatty acids, silicones (silicone oils), paraffins, nonionic surfactants, ester quats, glycerides of fatty acids (natural oils), waxes, mono-, di- or triglycerides, carbohydrates and/or polyalkylene glycols, preferably such ingredients having a melting point in the temperature range from 25° C. to 120° C.

Carbohydrates which may advantageously be used are here for example sugars, for example alpha-D-glucose monohydrate (melting point in the range 83-86° C.), alpha-D-galactose monohydrate (melting point in the range from 118-120° C.) or for example maltose monohydrate (melting point in the range 102-103° C.), to mention a few examples. Derivatives are also suitable, such as for example amino sugars, such as for example D-glucosamine (melting point a form: 88° C.), or such as for example deoxy sugars, such as for example rhamnose monohydrate (melting point 92-94° C.).

Suitable paraffins may be for example octadecane, nonadecane, eicosane, docosane, tricosane, tetracosane, pentacosane, hexacosane, heptacosane, octacosane, nonacosane or triacosane, to mention a few examples.

Suitable fatty alcohols may be for example 1-tridecanol, 1-tetradecanol, 1-pentadecanol, 1-hexadecanol, 1-heptadecanol, 1-octadecanol, 9-trans-octadecen-1-ol, 1-nonadecanol, 1-eicosanol, 1-heneicosanol, 1-docosanol, 13-cis-docosen-1-ol, 3-trans-docosen-1-ol, to mention just a few examples. These also include “wax alcohols”, namely fatty alcohols with approx. 24-36 carbon atoms, such as for example 1-triacontanol or melissyl alcohol. These also include unsaturated fatty alcohols such as for example elaidyl alcohol, erucic alcohol or brassidyl alcohol. These also include Guerbet alcohols, such as for example C₃₂H₆₆O or C₃₆H₇₄O. These also include alkanediols, such as for example 1,11-undecanediol or 1,12-dodecanediol.

Suitable nonionic surfactants may be for example fatty alcohol polyglycol ethers, such as for example C₁₄H₂₉—O—(CH₂CH₂O)₂H, C₁₀H₂₁—O—(CH₂CH₂O)₈H, C₁₂H₂₅—O—(CH₂CH₂O)₆H, C₁₄H₂₉—O—(CH₂CH₂O)₄H, C₁₆H₃₃—O—(CH₂₁CH₂O)₁₂H, C₁₈H₃₇—O—(CH₂CH₂O)₄H, to mention just a few examples.

Suitable fatty acids may be for example capric acid, undecanoic acid, lauric acid, tridecanoic acid, tetradecanoic acid, pentadecanoic acid, palmitic acid, margaric acid, stearic acid, nonadecanoic acid, arachidic acid, behenic acid, lignoceric acid, cerotic acid, crotonic acid, erucic acid, elaeostearic acid or melissic acid, to mention a few examples.

The esters of fatty acids may also be suitable, such as for example the methyl or ethyl esters of behenic acid or arachidic acid, to mention examples.

Mono-, di- or triglycerides are also suitable, for example the corresponding glycerides of lauric acid, palmitic acid or capric acid, to mention a few examples.

Suitable waxes may be natural waxes, such as for example carnauba wax, candelilla wax, esparto wax, guaruma wax, Japan wax, cork wax or montan wax, as well as animal waxes, such as for example beeswax, wool wax, shellac wax or spermaceti, as well as synthetic waxes, such as for example polyalkylene waxes or polyethylene glycol waxes, as well as chemically modified waxes, such as for example hydrogenated jojoba waxes or montan ester waxes.

The present invention further provides detergents or cleaning agents containing fluid-containing particles according to the invention. The term detergents or cleaning agents also covers textile pretreatment agents, post-treatment agents, treatment agents, conditioners, finishing agents and laundry rinse conditioners.

Preferred ingredients of the detergents or cleaning agents according to the invention are described in greater detail below. These ingredients mentioned below may be contained as such both in the fluid-containing particles according to the invention and in the detergents or cleaning agents according to the invention. Although generally reference is made below only to the detergents or cleaning agents according to the invention, all of the ingredients mentioned below may, as should here be explicitly clarified, be contained at least optionally in the fluid-containing particles according to the invention.

Anionic surfactants are preferably contained in the detergents or cleaning agents according to the invention. The anionic surfactants used may for example be those of the sulfonate and sulfate type. Surfactants of the sulfonate type which may here preferably be considered are C₉₋₁₃ alkyl benzene sulfonates, olefin sulfonates, i.e. mixtures of alkene and hydroxyalkane sulfonates and disulfonates, as are obtained, for example, from C₁₂₋₁₈ monoolefins with a terminal or internal double bond by sulfonation with gaseous sulfur trioxide and subsequent alkaline or acidic hydrolysis of the sulfonation products. Alkane sulfonates which are obtained from C₁₂₋₁₈ alkanes for example by sulfochlorination or sulfoxidation with subsequent hydrolysis or neutralization are also suitable. Likewise, the esters of α-sulfofatty acids (ester sulfonates) are also suitable, for example the α-sulfonated methyl esters of hydrogenated coconut, palm kernel or tallow fatty acids.

Anionic surfactants, in particular the above-stated anionic surfactants, may be contained in the fluid-containing particles according to the invention.

The content of anionic surfactants, preferably of the stated anionic surfactants, in the detergent or cleaning agent according to the invention, may vary widely, depending on the purpose to which the relevant agent is to be put. For instance, an agent according to the invention may contain very large quantities of anionic surfactant, preferably up to an order of magnitude of up to 40, 50 or 60 wt. % or more. Likewise, an agent according to the invention may contain only very small quantities of anionic surfactant, for example less than 15 or 10 wt. % or less than 5 wt. % or even less. Advantageously, however, anionic surfactants are contained in the agents according to the invention in quantities of from 1 to 40 wt. % and in particular 5 to 30 wt. %, concentrations of above 10 wt. % and even of above 15 wt. % being particularly preferred. According to one preferred embodiment, the detergent or cleaning agent according to the invention contains anionic surfactants, preferably in quantities of at least 0.1 wt. %, relative to the total detergent or cleaning agent. According to another preferred embodiment, the agent according to the invention contains no anionic surfactant.

In addition to the stated anionic surfactants, but also independently thereof, soaps may be contained in the detergents or cleaning agents according to the invention. Saturated fatty acid soaps are in particular suitable, such as the salts of lauric acid, myristic acid, palmitic acid, stearic acid, hydrogenated erucic acid and behenic acid and in particular soap mixtures derived from natural fatty acids, for example coconut, palm kernel or tallow fatty acids. The soap content of the agent amounts, independently of other anionic surfactants, preferably to no more than 3 wt. % and in particular 0.5 to 2.5 wt. %, relative to the total agent. According to another preferred embodiment, the agent according to the invention contains no soap.

Soaps, in particular the above-stated soaps, may be contained in the fluid-containing particles according to the invention.

The anionic surfactants and soaps may be present in the form of the sodium, potassium or ammonium salts thereof and as soluble salts of organic bases, such as mono-, di- or triethanolamine. Preferably they are present in the form of the sodium or potassium salts thereof, in particular in the form of the sodium salts. Anionic surfactants and soaps may also be produced in situ, by introducing the anionic surfactant acids and optionally fatty acids into the composition to be spray-dried, these then being neutralized by the alkalinity donors in the composition to be spray-dried.

Advantageously, nonionic surfactants may likewise be contained in the detergents or cleaning agents according to the invention. For example, their content may amount to up to 2 or 3 or 5 wt. %. Relatively large quantities of nonionic surfactant may also be contained, for example up to 5 wt. % or 10wt. % or 15wt. % or 20wt. %, 30wt. %, 40wt. %, 50wt. % or even more, if convenient. Sensible lower limit values may be 0.01, 0.1, 1, 2, 3 or 4 wt. %.

Preferably, however, the nonionic surfactants are contained in larger quantities, i.e. up to 50 wt. %, advantageously from 0.1 to 40 wt. %, particularly preferably from 0.5 to 30 and in particular from 2 to 25 wt. %, in each case relative to the total agent. According to a preferred embodiment, the detergent or cleaning agent according to the invention contains nonionic surfactants, preferably in quantities of at least 0.1 wt. %, relative to the total detergent or cleaning agent. According to another preferred embodiment, the agent according to the invention contains no nonionic surfactant.

Advantageously any nonionic surfactants known from the prior art may be contained in the agents according to the invention. Preferred nonionic surfactants are stated further below.

Nonionic surfactants, in particular the nonionic surfactants stated further below, may be contained in the fluid-containing particles according to the invention.

The detergent or cleaning agents according to the invention may preferably also contain cationic surfactants. Suitable cationic surfactants are for example quaternary surface-active compounds, in particular having an ammonium, sulfonium, phosphonium, iodonium or arsonium group. By using quaternary surface-active compounds having an antimicrobial action, it is possible to provide the agent with an antimicrobial action or to improve any antimicrobial action which it may already have due to other ingredients.

Particularly preferred cationic surfactants are the quaternary, in part antimicrobially active ammonium compounds (QACs; INCI Quaternary Ammonium Compounds) of the general formula (R^{I})(R^{II})(R^{III})(R^{IV})N⁺X⁻, in which R^{I} to R^{IV} represent identical or different C₁₋₂₂ alkyl residues, C₇₋₂₈ aralkyl residues or heterocyclic residues, with two or, in the case of aromatic incorporation as in pyridine, even three residues forming the heterocycle together with the nitrogen atom, for example a pyridinium or imidazolinium compound, and X⁻ is halide ions, sulfate ions, hydroxide ions or similar anions. To ensure optimum antimicrobial action, at least one of the residues preferably has a chain length of 8 to 18, in particular of 12 to 16, C atoms.

QACs may be produced by reacting tertiary amines with alkylating agents, such as for example methyl chloride, benzyl chloride, dimethyl sulfate, dodecyl bromide, and also ethylene oxide. Alkylation of tertiary amines with a long alkyl residue and two methyl groups is particularly easy, and the quaternization of tertiary amines with two long residues and a methyl group may be performed with the assistance of methyl chloride under mild conditions. Amines which have three long alkyl residues or hydroxy-substituted alkyl residues are not very reactive and are preferably quaternized with dimethyl sulfate.

Suitable QACs are for example benzalkonium chloride (N-alkyl-N,N-dimethylbenzylammonium chloride, CAS No. 8001-54-5), Benzalkon B (m,p-dichlorobenzyl dimethyl-C₁₂-alkylammonium chloride, CAS No. 58390-78-6), benzoxonium chloride (benzyldodecyl-bis-(2-hydroxyethyl)-ammonium chloride), cetrimonium bromide (N-hexadecyl-N,N-trimethylammonium bromide, CAS No. 57-09-0), benzetonium chloride (N,N-dimethyl-N-[2-[2-[p-(1,1,3,3-tetramethylbutyl)phenoxy]ethoxy]ethyl]-benzylammonium chloride, CAS No. 121-54-0), dialkyldimethylammonium chlorides such as di-n-decyldimethylammonium chloride (CAS No. 7173-51-5-5), didecyldimethylammonium bromide (CAS No. 2390-68-3), dioctyldimethylammonium chloride, 1-cetylpyridinium chloride (CAS No. 123-03-5) and thiazoline iodide (CAS No. 15764-48-1) and mixtures thereof. Preferred QACs are benzalkonium chlorides with C₈-C₁₈ alkyl residues, in particular C₁₂-C₁₄ alkylbenzyldimethylammonium chloride. One particularly preferred QAC is coconut pentaethoxymethylammonium methosulfate (INCI PEG-5 Cocomonium Methosulfate; Rewoquat® CPEM).

In order to avoid possible incompatibilities of the antimicrobial cationic surfactants with anionic surfactants which may be present in the detergent or cleaning agent according to the invention, the cationic surfactants used are those which are as highly compatible with anionic surfactants and/or are as slightly cationic as possible or, in one particular embodiment of the invention, cationic surfactants are completely omitted.

Further cationic surfactants, for instance quaternary ammonium compounds, are described further below in particular in connection with conditioners and softeners. These may also preferably be contained in the agents according to the invention.

Cationic surfactants may be contained in the fluid-containing particles according to the invention.

The detergents or cleaning agents according to the invention may contain one or more cationic surfactants, advantageously in quantities, relative to the total composition, of from 0 to 30 wt. %, still more advantageously of greater than 0 to 20 wt. %, preferably 0.01 to 10 wt. %, in particular 0.1 to 5 wt. %. Suitable minimum values may also be 0.5, 1, 2 or 3 wt. %. According to a preferred embodiment, the detergent or cleaning agent according to the invention contains cationic surfactants, preferably in quantities of at least 0.1 wt. %, relative to the total detergent or cleaning agent. According to another preferred embodiment, the agent according to the invention contains no cationic surfactant.

Likewise, the detergent or cleaning agent according to the invention may also contain amphoteric surfactants. These will be described in greater detail further below in particular in connection with conditioners and softeners.

The detergents or cleaning agents according to the invention may contain one or more amphoteric surfactants, advantageously in quantities, relative to the total composition, of from 0 to 30 wt. %, still more advantageously of greater than 0 to 20 wt. %, preferably of 0.01 to 10 wt. %, in particular of 0.1 to 5 wt. %. According to a preferred embodiment, the agent according to the invention contains no amphoteric surfactants.

Amphoteric surfactants may be contained in the fluid-containing particles according to the invention.

Further ingredients of the detergent or cleaning agents according to the invention and of the fluid-containing particles according to the invention may be inorganic and organic builder substances. The inorganic builder substances include water-insoluble or non-water-soluble ingredients, such as aluminosilicates and in particular zeolites.

In a preferred embodiment, the detergent or cleaning agent according to the invention does not contain any phosphate.

Provision may advantageously be made for the detergent or cleaning agent according to the invention to have a zeolite content of at least 10 wt. %, for example at least 15 wt. % or at least 20 wt. % or at least 30 wt. % or even more, for example at least 50 wt. %, relative to the total detergent or cleaning agent.

The detergent or cleaning agent according to the invention may contain soluble builders preferably in quantities of from 0.1 wt. % to 30 wt. %, preferably of 5 wt. % to 25 wt. % and particularly preferably of 10 wt. % to 20 wt. %, relative to the total weight of the agent, sodium carbonate being particularly preferred as a soluble builder. Provision may advantageously also be made for the agent according to the invention to contain less than 10 wt. %, for example less than 5 wt. % of soluble builders. According to another preferred embodiment, the agent according to the invention contains no soluble builders.

A finely crystalline, synthetic zeolite containing bound water which may be used is preferably zeolite A, and/or P. Zeolite MAP® (commercial product from Crosfield), for example, is particularly preferred as zeolite P. However, zeolite X and mixtures of A, X and/or P are also suitable. A co-crystallized sodium/potassium-aluminum silicate prepared from zeolite A and zeolite X, which is commercially available as VEGOBOND AX® (commercial product of Condea Augusta S.p.A.) is also of particular interest.

In one preferred embodiment of the invention, all the inorganic constituents contained should preferably be water-soluble. In these embodiments, builder substances other than the stated zeolites are therefore used.

Further suitable builder substances are polyacetals which may be obtained by reacting dialdehydes with polyolcarboxylic acids which comprise 5 to 7 C atoms and at least 3 hydroxyl groups. Preferred polyacetals are obtained from dialdehydes such as glyoxal, glutaraldehyde, terephthalaldehyde as well as mixtures thereof and from polyolcarboxylic acids such as gluconic acid and/or glucoheptonic acid.

Further suitable organic builder substances are dextrins, for example oligomers or polymers of carbohydrates, which may be obtained by partial hydrolysis of starches. Hydrolysis may be carried out in accordance with conventional, for example acid- or enzyme-catalysed processes. The hydrolysis products are preferably those with average molar masses in the range from 400 to 500000 g/mol. A polysaccharide with a dextrose equivalent (DE) in the range from 0.5 to 40, in particular from 2 to 30 is here preferred, DE being a conventional measure of the reducing action of a polysaccharide in comparison with dextrose, which has a DE of 100. Usable compounds are not only maltodextrins with a DE of between 3 and 20 and dry glucose syrups with a DE of between 20 and 37 but also “yellow” and “white” dextrins with higher molar masses in the range from 2000 to 30000 g/mol. One preferred dextrin is described in British patent application 94 19 091. The oxidized derivatives of such dextrins are the reaction products thereof with oxidizing agents which are capable of oxidizing at least one alcohol function of the saccharide ring to yield a carboxylic acid function.

Oxydisuccinates and other derivatives of disuccinates, preferably ethylenediamine disuccinate, are also further suitable cobuilders. Ethylenediamine-N,N′-disuccinate (EDDS) is here preferably used in the form of the sodium or magnesium salts thereof. Glycerol disuccinates and glycerol trisuccinates are also additionally preferred in this connection. Suitable utilization quantities are for example 3 to 15 wt. %, relative to the total detergent or cleaning agent.

Further usable organic cobuilders are, for example, acetylated hydroxycarboxylic acids or the salts thereof, which may optionally also be present in lactone form and which contain at least 4 carbon atoms and at least one hydroxy group and at most two acid groups.

Phosphonates are another class of substances with cobuilder properties. These in particular comprise hydroxyalkane- or aminoalkanephosphonates. Among hydroxyalkanephosphonates, 1-hydroxyethane-1,1-diphosphonate (HEDP) is of particular significance as a cobuilder. It is preferably used as a sodium salt, the disodium salt exhibiting a neutral reaction and the tetrasodium salt an alkaline (pH 9) reaction. Aminoalkanephosphonates which may preferably be considered are ethylenediaminetetramethylenephosphonate (EDTMP), diethylenetriaminepentamethylenephosphonate (DTPMP) as well as the higher homologs thereof. They are preferably used in the form of the sodium salts which exhibit a neutral reaction, for example as the hexasodium salt of EDTMP or as the hepta- and octasodium salt of DTPMP. From the class of phosphonates, HEDP is here preferably used as a builder. Aminoalkanephosphonates furthermore exhibit a pronounced heavy metal binding capacity. It may accordingly be preferred, especially if the detergents or cleaning agents also contain bleach, to use aminoalkanephosphonates, in particular DTPMP, or mixtures of the stated phosphonates.

In cases in which a phosphate content is tolerated, phosphates may also be used, in particular pentasodium triphosphate, optionally also pyrophosphates and orthophosphates, which primarily act as a precipitating agent for lime salts. Phosphates are predominantly used in dishwashing machines, but sometimes also in detergents.

Alkali metal phosphates is the general term for the alkali metal (in particular sodium and potassium) salts of the various phosphoric acids, which can be divided into metaphosphoric acids (HPO₃)ₙ and orthophosphoric acids H₃PO₄ in addition to representatives of a higher molecular weight. The phosphates here combine a number of advantages: they act as alkalinity donors, prevent lime deposits on parts of machinery or lime incrustation of fabrics and, moreover, contribute to cleaning performance.

In a preferred embodiment of the invention, carbonates and silicates are used in particular as inorganic builder substances.

Mention may here be made in particular of crystalline, layered sodium silicates of the general formula NaMSiₓO₂ₓ₊₁.yH₂O, M meaning sodium or hydrogen, x being a number from 1.6 to 4, preferably 1.9 to 4.0, and y a number from 0 to 20 and preferred values for x being 2, 3 or 4. Since, however, such crystalline silicates at least in part lose their crystalline structure in a spray drying procedure, crystalline silicates are preferably subsequently added to the direct or post-treated spray-drying product. Preferred crystalline phyllosilicates of the stated formula are those in which M denotes sodium and x assumes the values 2 or 3. In particular, both β- and δ-sodium disilicates Na₂Si₂O₅.yH₂O are preferred. Such compounds are commercially available for example under the name SKS® (Clariant). For instance, SKS-6® is predominantly a δ-sodium disilicate of the formula Na₂Si₂O₅.yH₂O, while SKS-7® is predominantly the β-sodium disilicate. Through reaction with acids (for example citric acid or carbonic acid), the δ-sodium disilicate yields kanemite NaHSi₂O₅.yH₂O, known commercially as SKS-9® or SKS-10® (Clariant). It may also be advantageous to use chemical modifications of these phyllosilicates. The alkalinity of the phyllosilicates may, for example, be suitably influenced in this manner. In comparison with δ-sodium disilicate, phyllosilicates doped with phosphate or with carbonate have modified crystal morphologies, dissolve more rapidly and exhibit an elevated calcium binding capacity compared with δ-sodium disilicate. For instance, phyllosilicates of the general empirical formula xNa₂O.ySiO₂.zP₂O₅ are known, in which the ratio x to y corresponds to a number from 0.35 to 0.6, the ratio x to z to a number from 1.75 to 1200 and the ratio y to z to a number from 4 to 2800. The solubility of phyllosilicates may also be increased by using particularly finely divided phyllosilicates. Compounds of crystalline phyllosilicates with other ingredients may also be used. Mention may be made in particular of compounds with cellulose derivatives, which have advantages in their disintegrating action, and compounds with polycarboxylates, for example citric acid, or polymeric polycarboxylates, for example copolymers of acrylic acid.

Preferred builder substances also include amorphous sodium silicates with an Na₂O:SiO₂ modulus of 1:2 to 1:3.3, preferably of 1:2 to 1:2.8 and in particular of 1:2 to 1:2.6, which exhibit secondary washing characteristics. For the purposes of the present invention, the term “amorphous” should also be taken to mean “X-ray amorphous”. This means that, in X-ray diffraction experiments, the silicates do not provide any sharp X-ray reflections, as are typical of crystalline substances, but at most one or more maxima of the scattered X-radiation, which have a width of two or more graduations of the diffraction angle. However, particularly good builder characteristics may very well be obtained if, in X-ray diffraction experiments, the silicate particles yield blurred or even sharp diffraction maxima. This should be interpreted to mean that the products comprise microcrystalline domains of a size of 10 to several hundred nm, values of up to at most 50 nm and in particular of up to at most 20 nm being preferred. Such “X-ray amorphous” silicates, which likewise exhibit dissolution retardation relative to conventional water glasses, are known. Compressed/compacted amorphous silicates, compounded amorphous silicates, and overdried, X-ray amorphous silicates are particularly preferred. The content of (X-ray) amorphous silicates in agents in particular not containing any zeolites preferably amounts 1 to 10 wt. %, which corresponds to a preferred embodiment of the invention.

Particularly preferred inorganic water-soluble builders are alkali metal carbonates and alkali metal bicarbonates, with sodium and potassium carbonate and in particular sodium carbonate being among preferred embodiments. The content of alkali metal carbonates in agents in particular not containing any zeolites may vary within a very wide range and amounts preferably to 1 to 50 wt. %, advantageously to 5 to 40 wt. %, in particular to 8 to 30 wt. %, the content of alkali metal carbonates conventionally being higher than that of (X-ray) amorphous silicates. According to another preferred embodiment, the detergent or cleaning agent according to the invention contains no alkali metal carbonates.

Organic matrix materials which may be used are for example the polycarboxylic acids usable in the form of the alkali metal and in particular sodium salts thereof, such as citric acid, adipic acid, succinic acid, glutaric acid, tartaric acid, saccharic acids, aminocarboxylic acids, nitrilotriacetic acid (NTA), provided that there are no objections to such use on environmental grounds. Preferred salts are the salts of polycarboxylic acids such as citric acid, adipic acid, succinic acid, glutaric acid, tartaric acid, saccharic acids and mixtures of these. The acids themselves may also be used. Apart from their builder action, the acids typically also have the property of an acidifying component and so also serve, as for example in the granules according to the invention, to establish a lower and gentler pH value for detergent or cleaning agents. Citric acid, succinic acid, glutaric acid, adipic acid, gluconic acid and any desired mixtures of these may in particular be mentioned.

Further suitable organic builders are polymeric polycarboxylates, these being for example the alkali metal salts of polyacrylic acid or polymethacrylic acid, for example those with a relative molecular mass of 500 to 70000 g/mol. The molar masses indicated for polymeric polycarboxylates comprise for the purposes of this document weight-average molar masses Mw of the respective acid form, these having basically been determined by means of gel permeation chromatography (GPC), a UV detector having been used. Measurement was here made relative to an external polyacrylic acid standard, which supplies realistic molecular weight values as a result of its structural relationship to the polymers under investigation. These values differ markedly from the molecular weight values at which polystyrene sulfonic acids are used as a standard. The molar masses measured relative to polystyrenesulfonic acids are generally markedly higher than the molar masses indicated in the present document.

The detergents or cleaning agents according to the invention may also contain polymers. Suitable polymers comprise in particular polyacrylates, which preferably have a molecular mass of 2000 to 20000 g/mol. Due to their superior solubility, the short-chain polyacrylates from this group may in turn be preferred, these having molar masses of from 2000 to 10000 g/mol, and particularly preferably of from 3000 to 5000 g/mol.

Also suitable are copolymeric polycarboxylates, in particular those of acrylic acid with methacrylic acid and acrylic acid or methacrylic acid with maleic acid. Copolymers of acrylic acid with maleic acid containing 50 to 90 wt. % acrylic acid and 50 to 10 wt. % maleic acid have proven particularly suitable. Their relative molecular mass, relative to free acids, amounts in general to 2000 to 70000 g/mol, preferably 20000 to 50000 g/mol and in particular 30000 to 40000 g/mol.

The organic builder substance content of detergents or cleaning agents may vary widely. Contents of 2 to 20 wt. % are preferred, with contents of at most 10 wt. % finding particular favor. According to another preferred embodiment, the detergent or cleaning agent according to the invention contains no organic builder substances.

It should be mentioned at this point that, unless otherwise stated, the weight percentage values relate in each case to the total detergent or cleaning agent.

The detergents or cleaning agents according to the invention may comprise components from the classes of graying inhibitors (soil carriers), neutral salts and/or the textile-softening auxiliaries (for example cationic surfactants), this being preferred.

Graying inhibitors have the task of keeping soiling which has been dissolved off the fibers in suspension in the liquor and so preventing redeposition of the soiling. Water-soluble colloids of a mainly organic nature are suitable for this purpose, for example the water-soluble salts of polymeric carboxylic acids, size, gelatin, salts of ether carboxylic acids or ether sulfonic acids of starch or cellulose or salts of acidic sulfuric acid esters of cellulose or starch. Water-soluble polyamides containing acidic groups are also suitable for this purpose. Soluble starch preparations and starch products other than those mentioned above may furthermore be used, for example degraded starch, aldehyde starches etc. Polyvinylpyrrolidone may also be used. However, cellulose ethers, such as carboxymethylcellulose (Na salt), methylcellulose, hydroxyalkylcellulose and mixed ethers, such as methylhydroxyethylcellulose, methylhydroxypropylcellulose, methylcarboxymethylcellulose and mixtures thereof, together with polyvinylpyrrolidone, are preferably used, for example in quantities preferably of 0.1 to 5 wt. %, relative to the detergents or cleaning agents.

A typical example which may be mentioned of a suitable representative of neutral salts is sodium sulfate. This may be used in quantities of for example 0 to 60 wt. %, preferably of 2 to 45 wt. %.

Suitable softeners, which are described in greater detail further below, are for example swellable phyllosilicates of the type comprising corresponding montmorillonites, for example bentonite, as well as cationic surfactants.

The water content in the detergent or cleaning agent amounts preferably to 0 to less than 30 wt. % and in particular to 0.5 to less than 20 wt. %, values of at most 15 wt. %, at most 10 wt. %, at most 5 wt. %, at most 3 wt. % or at most 2 wt. % being particularly preferred. Any water adhering to optionally present aluminosilicates such as zeolites was here not included in these calculations.

According to a preferred embodiment, the detergent or cleaning agent according to the invention is substantially solid in form, being preferably present in pulverulent, pressed or granular form.

Nonionic surfactants, which may preferably be contained in the detergent or cleaning agent, are described in greater detail below. These nonionic surfactants may also be applied for example in a post-treatment step to the particulate detergent or cleaning agent. It goes without saying that any nonionic surfactants may, however, advantageously be contained directly in the detergent or cleaning agent according to the invention.

Alkoxylated, advantageously ethoxylated, in particular primary alcohols with preferably 8 to 18 C atoms and on average 1 to 12 mol of ethylene oxide (EO) per mol of alcohol, in which the alcohol residue may be linear or preferably methyl-branched in position 2 or may contain linear and methyl-branched residues in the mixture, as they are usually present in oxo alcohol residues, are preferably used as nonionic surfactants. In particular, however, alcohol ethoxylates with linear residues prepared from alcohols of natural origin with 12 to 18 C atoms, for example from coconut, palm, palm kernel, tallow fat or oleyl alcohol, and on average 2 to 8 EO per mol of alcohol are preferred. The preferred ethoxylated alcohols include for example C₁₂-C₁₄ alcohols with 3 EO to 6 EO, C₉-C₁₁ alcohols with 7 EO, C₁₃-C₁₅ alcohols with 3 EO, 5 EO, 7 EO or 8 EO, C₁₄-C₁₅ alcohols with 4 EO, 5 EO, 7 EO or 9 EO, C₁₂-C₁₈ alcohols with 3 EO, 5 EO or 7 EO and mixtures of these, such as mixtures of C₁₂-C₁₄ alcohol with 3 EO and C₁₂-C₁₈ alcohol with 7 EO. The stated degrees of ethoxylation are statistical averages which, for a specific product, may be an integer or a fractional number.

Preferred alcohol ethoxylates have a narrow homolog distribution (narrow range ethoxylates, NRE). In addition to these nonionic surfactants, fatty alcohols with more than 12 EO may also be used. Examples of these are (tallow) fatty alcohols with 14 EO, 16 EO, 20 EO, 25 EO, 30 EO or 40 EO.

Preferred nonionic surfactants are one or more unbranched or branched, saturated or unsaturated C₁₀₋₂₂ alcohols alkoxylated with ethylene (EO) and/or propylene oxide (PO) and with a degree of alkoxylation of up to 30, preferably ethoxylated C₁₀₋₁₈ fatty alcohols with a degree of ethoxylation of less than 30, preferably of 1 to 20, in particular of 1 to 12, particularly preferably of 1 to 8, extremely preferably of 2 to 5, for example C₁₂₋₁₄ fatty alcohol ethoxylates with 2, 3 or 4 EO or a mixture of C₁₂₋₁₄ fatty alcohol ethoxylates with 3 and 4 EO in a weight ratio of 1:1 or isotridecyl alcohol ethoxylate with 5, 8 or 12 EO.

Alkyl glycosides of the general formula RO(G)ₓ, in which R means a primary linear or methyl-branched aliphatic residue, in particular methyl-branched in position 2, with 8 to 22, preferably 12 to 18 C atoms and G is the symbol which denotes a glycose unit with 5 or 6 C atoms, preferably glucose, may moreover be used as further nonionic surfactants. The degree of oligomerization x, which indicates the distribution of monoglycosides and oligoglycosides, is any desired number between 1 and 10; x is preferably 1.1 to 1.4.

A further class of nonionic surfactants which may preferably be used, which may be used either as the sole nonionic surfactant or in combination with other nonionic surfactants, in particular together with alkoxylated fatty alcohols and/or alkyl glycosides, are alkoxylated, preferably ethoxylated or ethoxylated and propoxylated fatty acid alkyl esters, preferably with 1 to 4 carbon atoms in the alkyl chain, in particular fatty acid methyl esters, C₁₂-C₁₈ fatty acid methyl esters with an average of 3 to 15 EO, in particular with an average of 5 to 12 EO, may particularly preferably be used, for example.

Nonionic surfactants of the amine oxide type, for example N-coconut alkyl-N,N-dimethylamine oxide and N-tallow alcohol-N,N-dihydroxyethylamine oxide, and the fatty acid alkanolamide type may also be suitable. The quantity of these nonionic surfactants preferably amounts to no more than that of the ethoxylated fatty alcohols, in particular no more than half the quantity thereof.

Also suitable are alkoxylated amines, advantageously ethoxylated and/or propoxylated, in particular primary and secondary amines with preferably 1 to 18 C atoms per alkyl chain and on average 1 to 12 mol of ethylene oxide (EO) and/or 1 to 10 mol of propylene oxide (PO) per mol of amine.

In the case of agents according to the invention which are particularly suitable for machine dishwashing, in particular dishwashing detergents in the form of molded tablets, such as tabs, any surfactants may in principle be considered as surfactants. However, precisely the above-described nonionic surfactants are preferred for this intended application, here above all the low-foam nonionic surfactants. Alkoxylated alcohols, in particular ethoxylated and/or propoxylated alcohols, are particularly preferred. The person skilled in the art generally understands the term alkoxylated alcohols to mean the reaction products of alkylene oxide, preferably ethylene oxide, with alcohols, preferably for the purposes of the present invention the relatively long chain alcohols C₁₀ to C₁₈, preferably from C₁₂ to C₁₆, such as C₁₁, C₁₂, C₁₃, C₁₄, C₁₅, C₁₆, C₁₇ and C₁₈ alcohols. As a rule, a complex mixture of addition products of different degrees of ethoxylation is obtained from n mol of ethylene oxide and one mol of alcohol, depending on the reaction conditions. A further embodiment involves the use of mixtures of alkylene oxides, preferably a mixture of ethylene oxide and propylene oxide. It is also possible, if desired, to arrive through final etherification with short-chain alkyl groups, such as preferably the butyl group, at the class of substances comprising “closed” alcohol ethoxylates, which may likewise be used for the purposes of the invention. Very particularly preferred for the purposes of the present invention are highly ethoxylated fatty alcohols or mixtures thereof with end group-terminated fatty alcohol ethoxylates.

Advantageously, the detergents or cleaning agents according to the invention may also contain foam inhibitors, for example foam-inhibiting paraffin oil or foam-inhibiting silicone oil, for example dimethylpolysiloxane. The use of mixtures of these active ingredients is also possible. Feasible additives which are solid at room temperature are, in particular in the case of the stated foam-inhibiting active ingredients, paraffin waxes, silicas, which may also be hydrophobized in known manner, and bisamides derived from C₂₋₇ diamines and C₁₂₋₂₂ carboxylic acids.

UV absorbers may advantageously be contained in the detergent or cleaning agent in quantities of from 0.01 wt. % to 5 wt. %, preferably of from 0.03 wt. % to 1 wt. %. They may also subsequently be admixed with the detergent or cleaning agent, for example together with other substances.

Preferably, the detergents or cleaning agents according to the invention may also be present as tablets or shaped articles. “Tablet” or “shaped article” is used for the purposes of the present application to denote dimensionally stable, solid bodies, irrespective of their manner of production. Such bodies may be produced for example by crystallization, casting, injection molding, reactive or thermal sintering, (co)extrusion, prilling, pastillization or compacting methods such as calendering or tabletting. For the purposes of the present application, production of “tablets” or “shaped articles” by tabletting is particularly preferred. The tablet thus preferably consists of compression-molded, particulate material.

Detergents or cleaning agents according to the invention may preferably contain disintegration auxiliaries. Suitable swellable disintegration auxiliaries are for example bentonites or other swellable silicates. Synthetic polymers, in particular the superabsorbers or crosslinked polyvinylpyrrolidone used in the hygiene sector, may also be used.

In addition, the agents according to the invention may be conditioners and contain appropriate components. The term conditioning should be understood for the purposes of the present invention preferably to mean finishing treatment for textiles, materials and fabrics. Conditioning lends the textiles positive properties, such as for example improved soft handle, elevated luster and color brightness, an improved odor impression, reduced felting, easier ironing by reducing surface slip properties, reduced creasing and static charge and dye transfer inhibition in the case of dyed textiles.

To improve soft handle and finishing properties, the agents according to the invention may comprise softener components. Examples of such compounds are quaternary ammonium compounds, cationic polymers and emulsifiers, as are used in hair care products and also in textile finishing agents. These softening compounds, which will also be described in greater detail below, may be contained in any agents according to the invention, but in particular in conditioners or in agents intended to have a softening action.

Suitable examples are quaternary ammonium compounds of the formulae (III) and (IV),

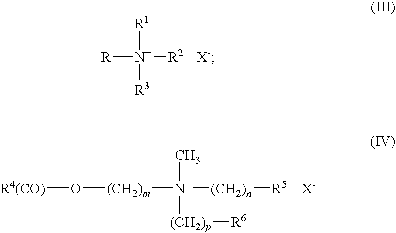

with, in (III), R and R¹ denoting an acyclic alkyl residue with 12 to 24 carbon atoms, R² denoting a saturated C₁-C₄ alkyl or hydroxyalkyl residue, R³ being either identical to R, R¹ or R² or denoting an aromatic residue. X⁻ denotes alternatively a halide, methosulfate, methophosphate or phosphate ion and mixtures of these. Examples of cationic compounds of the formula (III) are didecyldimethylammonium chloride, ditallowdimethylammonium chloride or dihexadecylammonium chloride.

Compounds of the formula (IV) are known as “ester quats”. Ester quats are distinguished by excellent biodegradability. R⁴ here denotes an aliphatic alkyl residue with 12 to 22 carbon atoms with 0, 1, 2 or 3 double bonds; R⁵ denotes H, OH, or O(CO)R⁷, R⁶, independently of R⁵, denotes H, OH, or O(CO)R⁸, R⁷ and R⁸ mutually independently in each case denoting an aliphatic alk(en)yl residue with 12 to 22 carbon atoms with 0, 1, 2 or 3 double bonds. m, n and p may in each case mutually independently have the value 1, 2 or 3. X⁻ may be alternatively a halide, methosulfate, methophosphate or phosphate ion and mixtures of these. Preferred compounds are those which for R⁵ contain the group O(CO)R⁷ and for R⁴ and R⁷ contain alkyl residues with 16 to 18 carbon atoms. Particularly preferred compounds are those in which R⁶ additionally denotes OH. Examples of compounds of the formula (IV) are methyl-N-(2-hydroxyethyl)-N,N-di(tallowacyloxyethyl)ammonium methosulfate, bis-(palmitoyl)-ethylhydroxyethyl methylam monium methosulfate or methyl-N,N-bis(acyloxyethyl)-N-(2-hydroxyethyl)ammonium methosulfate. If quaternized compounds of the formula (IV) which comprise unsaturated alkyl chains are used, preferred acyl groups are those whose corresponding fatty acids exhibit an iodine value of between 5 and 80, preferably of between 10 and 60 and in particular of between 15 and 45 and which have a cis/trans isomer ratio (in wt. %) of greater than 30:70, preferably of greater than 50:50 and in particular of greater than 70:30. Conventional commercial examples are the methylhydroxyalkyldialkoyloxyalkylammonium methosulfates distributed by Stepan under the trademark Stepantex® or the Cognis products known by the name Dehyquart® or the Goldschmidt-Witco products known by the name Rewoquat®. Further preferred compounds are the diester quats of the formula (V), which are obtainable under the name Rewoquat® W 222 LM or CR 3099 and, in addition to softness, also provide stability and color protection.

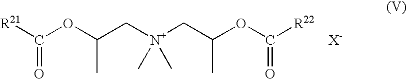

R²¹ and R²² here mutually independently in each case denote an aliphatic residue with 12 to 22 carbon atoms with 0, 1, 2 or 3 double bonds. In addition to the above-described quaternary compounds, other known compounds may also be used, such as for example quaternary imidazolinium compounds of the formula (VI),

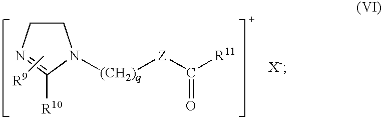

R⁹ denoting H or a saturated alkyl residue with 1 to 4 carbon atoms, R¹⁰ and R¹¹ mutually independently in each case denoting an aliphatic, saturated or unsaturated alkyl residue with 12 to 18 carbon atoms, R¹⁰ alternatively possibly also denoting O(CO)R²⁰, R²⁰ meaning an aliphatic, saturated or unsaturated alkyl residue with 12 to 18 carbon atoms, and Z meaning an NH group or oxygen and X⁻ being an anion. q may assume integral values between 1 and 4. Further suitable quaternary compounds are described by the formula (VII),

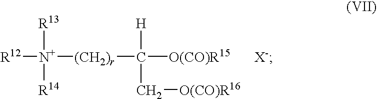

R¹², R¹³ and R¹⁴ mutually independently denoting a C₁₋₄ alkyl, alkenyl or hydroxyalkyl group, R¹⁵ and R¹⁶, in each case independently selected, representing a C₈₋₂₈ alkyl group and r being a number between 0 and 5.

In addition to the compounds of the formulae (III) and (IV), short-chain, water-soluble, quaternary ammonium compounds may also be used, such as trihydroxyethylmethylammonium methosulfate or alkyltrimethylammonium chloride, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride.

Protonated alkylamine compounds which exhibit a softening action and the non-quaternized, protonated precursors of cationic emulsifiers are also suitable.

Quaternized protein hydrolysates are further cationic compounds which are usable according to the invention.

Suitable cationic polymers include polyquaternium polymers, as are mentioned in the CTFA Cosmetic Ingredient Dictionary (The Cosmetic, Toiletry and Fragrance Association, Inc., 1997), in particular polyquaternium-6, polyquaternium-7, polyquaternium-10 polymers (Ucare Polymer IR 400; Amerchol) also known as Merquats, polyquaternium-4 copolymers, such as graft copolymers with a cellulose skeleton and quaternary ammonium groups, which are attached via allyldimethylammonium chloride, cationic cellulose derivatives, such as cationic guar, such as guarhydroxypropyltriammonium chloride, and similar quaternized guar derivatives (for example Cosmedia Guar, manufacturer: Cognis GmbH), cationic quaternary sugar derivatives (cationic alkyl polyglucosides), for example the commercial product Glucquat®100, according to CTFA nomenclature a “Lauryl Methyl Gluceth-10 Hydroxypropyl Dimonium Chloride”, copolymers of PVP and dimethylamino methacrylate, copolymers of vinylimidazole and vinylpyrrolidone, aminosilicone polymers and copolymers.

Alkylated quaternary ammonium compounds, at least one alkyl chain of which is interrupted by an ester group and/or amido group, in particular N-methyl-N(2-hydroxyethyl)-N,N-(ditallowacyloxyethyl)ammonium methosulfate are particularly preferred.

Nonionic softeners which may primarily be considered are polyoxyalkylene glycerol alkanoates, polybutylenes, long-chain fatty acids, ethoxylated fatty acid ethanolamides, alkyl polyglycosides, in particular sorbitan mono-, di- and triesters and fatty acid esters of polycarboxylic acids.

An agent according to the invention, preferably a conditioner, may contain softeners in quantities of 0.1 to 80 wt. %, conventionally of 0.1 to 70 wt. %, preferably of 0.2 to 60 wt. % and in particular of 0.5 to 40 wt. %, in each case relative to the entire agent.

Conditioners according to the invention may preferably contain one or more anionic surfactants, in particular those which have already been described further above.

Conditioners according to the invention may preferably contain one or more nonionic surfactants, in particular those which have already been described further above.

The detergents or cleaning agents according to the invention may preferably also contain amphoteric surfactants. In addition to numerous mono- to tri-alkylated amine oxides, betaines are a significant class.

In a preferred embodiment, the detergent or cleaning agent according to the invention, such as in particular the conditioner, may optionally contain one or more complexing agent(s).

Complexing agents (INCI Chelating Agents), which are also known as sequestrants, are ingredients which are capable of complexing and inactivating metal ions, for example in order to prevent their disadvantageous effects on the stability or appearance, for example cloudiness, of the agents. On the one hand, it is important to complex the calcium and magnesium ions of water hardness which are incompatible with numerous ingredients. Complexation of heavy metal ions such as iron or copper delays oxidative decomposition of the finished agent.

Suitable complexing agents according to INCI nomenclature are those which are for example described in greater detail in the International Cosmetic Ingredient Dictionary and Handbook: Aminotrimethylene Phosphonic Acid, Beta-Alanine Diacetic Acid, Calcium Disodium EDTA, Citric Acid, Cyclodextrin, Cyclohexanediamine Tetraacetic Acid, Diammonium Citrate, Diammonium EDTA, Diethylenetriamine Pentamethylene Phosphonic Acid, Dipotassium EDTA, Disodium Azacycloheptane Diphosphonate, Disodium EDTA, Disodium Pyrophosphate, EDTA, Etidronic Acid, Galactaric Acid, Gluconic Acid, Glucuronic Acid, HEDTA, Hydroxypropyl Cyclodextrin, Methyl Cyclodextrin, Pentapotassium Triphosphate, Pentasodium Aminotrimethylene Phosphonate, Pentasodium Ethylenediamine Tetramethylene Phosphonate, Pentasodium Pentetate, Pentasodium Triphosphate, Pentetic Acid, Phytic Acid, Potassium Citrate, Potassium EDTMP, Potassium Gluconate, Potassium Polyphosphate, Potassium Trisphosphonomethylamine Oxide, Ribonic Acid, Sodium Chitosan Methylene Phosphonate, Sodium Citrate, Sodium Diethylenetriamine Pentamethylene Phosphonate, Sodium Dihydroxyethylglycinate, Sodium EDTMP, Sodium Gluceptate, Sodium Gluconate, Sodium Glycereth-1 Polyphosphate, Sodium Hexametaphosphate, Sodium Metaphosphate, Sodium Metasilicate, Sodium Phytate, Sodium Polydimethylglycinophenolsulfonate, Sodium Trimetaphosphate, TEA-EDTA, TEA-Polyphosphate, Tetrahydroxyethyl Ethylenediamine, Tetrahydroxypropyl Ethylenediamine, Tetrapotassium Etidronate, Tetrapotassium Pyrophosphate, Tetrasodium EDTA, Tetrasodium Etidronate, Tetrasodium Pyrophosphate, Tripotassium EDTA, Trisodium Dicarboxymethyl Alaninate, Trisodium EDTA, Trisodium HEDTA, Trisodium NTA and Trisodium Phosphate.

Preferred complexing agents are tertiary amines, in particular tertiary alkanolamines (aminoalcohols). The alkanolamines have both amino and hydroxy and/or ether groups as functional groups. Particularly preferred tertiary alkanolamines are triethanolamine and tetra-2-hydroxypropylethylenediamine (N,N,N′,N′-tetrakis-(2-hydroxypropyl)ethylenediamine). Particularly preferred combinations of tertiary amines with zinc ricinoleate and one or more ethoxylated fatty alcohols as nonionic solubilizing agents and optionally solvents are described in the prior art.

One particularly preferred complexing agent is etidronic acid (1-hydroxyethylidene-1,1-diphosphonic acid, 1-hydroxyethylene-1,1-diphosphonic acid, HEDP, acetophosphonic acid, INCI Etidronic Acid) including the salts thereof. In a preferred embodiment, the detergent or cleaning agent according to the invention accordingly contains etidronic acid and/or one or more the salts thereof as complexing agent.

In one particular embodiment, the detergent or cleaning agent according to the invention contains a complexing agent combination of one or more tertiary amines and one or more further complexing agents, preferably one or more complexing agent acids or the salts thereof, in particular of triethanolamine and/or tetra-2-hydroxypropylethylendiamine and etidronic acid and/or one or more of the salts thereof.

The detergent or cleaning agent according to the invention, such as in particular conditioner, advantageously contains complexing agents in a quantity of conventionally 0 to 20 wt. %, preferably of 0.1 to 15 wt. %, in particular of 0.5 to 10 wt. %, particularly preferably of 1 to 8 wt. %, extremely preferably of 1.5 to 6 wt. %, relative to the entire agent.

ln a further preferred embodiment, the detergent or cleaning agent according to the invention, such as in particular conditioner, optionally contains one or more enzymes.

Enzymes which may in particular be considered are those from the classes of hydrolases such as proteases, esterases, lipases or lipolytically active enzymes, amylases, cellulases or other glycosylhydrolases and mixtures of the stated enzymes. In laundry, all these hydrolases contribute to the removal of stains such as those containing protein, fat or starch and of graying. By removing pilling and microfibrils, cellulases and other glycosylhydrolases may furthermore contribute to color retention and to increasing textile softness. Oxyreductases may also be used for bleaching or for inhibiting color transfer.

The enzymes may be adsorbed on support materials or be embedded in coatings in order to protect them from premature breakdown. The proportion of enzymes, enzyme mixtures or enzyme pellets may for example amount to approx. 0.1 to 5 wt. %, preferably to 0.12 to approx 2 wt. %, relative to the entire agent.

The detergent or cleaning agents according to the invention (for example conditioners) may optionally contain bleaching agents. Among those compounds acting as bleaching agents which release H₂O₂ in water, sodium percarbonate, sodium perborate tetrahydrate and sodium perborate monohydrate are of particular significance.

The bleaching agents may preferably be coated in order to protect them from premature breakdown.

Dyes may be used in the detergent or cleaning agent according to the invention, the selected quantity of one or more dyes ideally being such that no visible residues remain after use of the agent. The agent according to the invention preferably contains no dyes.

The agent according to the invention may preferably contain one or more antimicrobial active ingredients or preservatives conventionally in a quantity of 0.0001 to 3 wt. %, preferably of 0.0001 to 2 wt. %, in particular of 0.0002 to 1 wt. %, particularly preferably of 0.0002 to 0,2 wt. %, extremely preferably of 0.0003 to 0.1 wt. %.

In relation to antimicrobial active ingredients or preservatives, a distinction is drawn, depending on the antimicrobial spectrum and mechanism of action, between bacteriostatics and bactericides, fungistatics and fungicides etc. Significant substances from these groups are, for example, benzalkonium chlorides, alkylarylsulfonates, halophenols and phenol mercuriacetate. The terms antimicrobial action and antimicrobial active ingredient have the conventional meaning for the purposes of the teaching according to the invention. Suitable antimicrobial active ingredients are preferably selected from the groups comprising alcohols, amines, aldehydes, antimicrobial acids or the salts thereof, carboxylic acid esters, acid amides, phenols, phenol derivatives, diphenyls, diphenyl alkanes, urea derivatives, oxygen acetals and formals, nitrogen acetals and formals, benzamidines, isothiazolines, phthalimide derivatives, pyridine derivatives, antimicrobial surface-active compounds, guanidines, antimicrobial amphoteric compounds, quinolines, 1,2-dibromo-2,4-dicyanobutane, iodo-2-propylbutyl carbamate, iodine, iodophores, peroxo compounds, halogen compounds and any desired mixtures of the above.

Furthermore, the detergents or cleaning agents according to the invention may optionally contain ironing aids for improving the water absorption capacity and re-wettability of the treated textiles and for making it easier to iron the treated textiles. Silicone derivatives may for example be used in the formulations. These additionally improve rinsing out of the detergent formulations due to their foam-inhibiting properties. Preferred silicone derivatives are for example polydialkyl or alkylaryl siloxanes, in which the alkyl groups comprise one to five C atoms and are wholly or partially fluorinated. Preferred silicones are polydimethylsiloxanes, which may optionally be derivatized and are then amino-functional or quaternized or comprise Si—OH, Si—H and/or Si—Cl bonds. The viscosities of the preferred silicones at 25° C. lie in the range between 100 and 100,000 mPa s, it being possible to use the silicones in quantities of between 0.2 and 5 wt. %, relative to the total agent.

It should once again be pointed out that all the ingredients mentioned here in connection with the detergents or cleaning agents according to the invention may be contained in the fluid-containing particles according to the invention.

A pulverulent (all-in-one) detergent according to the invention may preferably contain for example components selected inter alia from the following:
anionic surfactants, such as for example alkylbenzenesulfonate, alkyl sulfate, in quantities of advantageously 5-30 wt. %, preferably 8-15 wt. %, in particular 15-20 wt. %,
nonionic surfactants, such as for example fatty alcohol polyglycol ether, alkyl polyglucoside, fatty acid glucamide, advantageously in quantities of 0.1-20 wt. %, preferably 2-15 wt. %, in particular 6-11 wt. %,
builders, such as for example zeolite, polycarboxylate, sodium citrate, for example in quantities of 5-60 wt. %, preferably in quantities of 10-55 wt. %, in particular of 15-40 wt. %,
alkalis, such as for example sodium carbonate, advantageously in quantities of 1-30 wt. %, preferably of 2-25 wt. %, in particular of 5-20 wt. %,
bleaching agents, such as for example sodium perborate, sodium percarbonate, advantageously in quantities of 5-25 wt. %, preferably of 10-20 wt. %,
corrosion inhibitors, such as for example sodium silicate, advantageously in quantities of 1-6 wt. %, preferably of 2-5 wt. %, in particular of 3-4 wt. %,
stabilizers such as for example phosphonates, advantageously in quantities of 0-1 wt. %,
foam inhibitors, such as for example soap, silicone oils, paraffins, advantageously in quantities of 0.1-4 wt. %, preferably of 0.2-2 wt. %, in particular of 1-3 wt. %,
enzymes, such as for example proteases, amylases, cellulases, lipases, advantageously in quantities of 0.1-2 wt. %, preferably of 0.2-1 wt. %, in particular of 0.3-0.8 wt. %,
graying inhibitors, such as for example carboxymethylcellulose, advantageously in quantities of 0-1 wt. %,
discoloration inhibitors, such as for example polyvinylpyrrolidone derivatives, advantageously in quantities of 0-2 wt. %,
- fillers, such as for example sodium sulfate, advantageously in quantities of 0-20 wt. %,
optical brighteners, such as for example stilbene derivative, biphenyl derivative, advantageously in quantities of 0.1-0.4 wt. %, in particular of 0.1-0.3 wt. %,
fragrances
water
soap
bleaching activators
cellulose derivatives
soil repellents

The detergents or cleaning agents according to the invention may preferably also be perfumed with perfume oil (odoriferous substances, fragrances).

In a preferred embodiment, the detergent or cleaning agent according to the invention contains specific minimum values of perfume oil, namely at least 0.01 wt. %, advantageously at least 0.1 wt. %, considerably advantageously at least 0.15 wt. %, more advantageously at least 0.2 wt. %, even more advantageously at least 0.25 wt. %, still more advantageously at least 0.3 wt. %, very advantageously at least 0.35 wt. %, particularly advantageously at least 0.4 wt. %, very particularly advantageously at least 0.45 wt. %, considerably advantageously at least 0.5 wt. %, very considerably advantageously at least 0.55 wt. %, extremely advantageously at least 0.6 wt. %, extremely advantageously at least 0.65 wt. %, extremely advantageously at least 0.7 wt. %, exceptionally advantageously at least 0.75 wt. %, extraordinarily advantageously at least 0.8 wt. %, extraordinarily advantageously at least 0.85 wt. %, in particular at least 0.9 wt. % of perfume oil, relative to the total detergent or cleaning agent.

The present invention also provides use of the fluid-containing particles according to the invention for room, vehicle or closet fragrancing, in particular in the form of fragrancing sachets.

The present invention also provides use of the fluid-containing particles for fragrancing objects, preferably detergents, washing and cleaning machines, dry laundry and packaging.

The present invention also provides use of the fluid-containing particles for fragrancing textiles during the washing or drying process, preferably performed by machine.

The present invention also provides use of the fluid-containing particles for protecting the fluid contained therein, in particular perfume, against an aggressive medium, in particular a detergent matrix.

While particular embodiments of the present invention have been illustrated and described, it would be obvious to those skilled in the art that various other changes and modifications can be made without departing from the spirit and scope of the invention.

Other than where otherwise indicated, or where required to distinguish over the prior art, all numbers expressing quantities of ingredients herein are to be understood as modified in all instances by the term “about”. As used herein, the words “may” and “may be” are to be interpreted in an open-ended, non-restrictive manner. At minimum, “may” and “may be” are to be interpreted as definitively including, but not limited to, the composition, structure, or act recited.

As used herein, and in particular as used herein to define the elements of the claims that follow, the articles “a” and “an” are synonymous and used interchangeably with “at least one” or “one or more,” disclosing or encompassing both the singular and the plural, unless specifically defined herein otherwise. The conjunction “or” is used herein in both in the conjunctive and disjunctive sense, such that phrases or terms conjoined by “or” disclose or encompass each phrase or term alone as well as any combination so conjoined, unless specifically defined herein otherwise.

The description of a group or class of materials as suitable or preferred for a given purpose in connection with the invention implies that mixtures of any two or more of the members of the group or class are equally suitable or preferred. Description of constituents in chemical terms refers unless otherwise indicated, to the constituents at the time of addition to any combination specified in the description, and does not necessarily preclude chemical interactions among the constituents of a mixture once mixed. Steps in any method disclosed or claimed need not be performed in the order recited, except as otherwise specifically disclosed or claimed.

Changes in form and substitution of equivalents are contemplated as circumstances may suggest or render expedient. Although specific terms have been employed herein, such terms are intended in a descriptive sense and not for purposes of limitation.

The following Examples further illustrate the preferred embodiments within the scope of the present invention, but are not intended to be limiting thereof. It is understood that the examples and embodiments described herein are for illustrative purposes only and that various modifications or changes in light thereof will be suggested to one skilled in the art without departing from the scope of the present invention. The appended claims therefore are intended to cover all such changes and modifications that are within the scope of this invention.

### Examples

Composition A

A tower powder containing zeolite was granulated with a 4.6 wt. %, aqueous carboxymethylcellulose (CMC) solution. After drying, the resultant agglomerate comprised 94.98 wt. % of tower powder, 0.19 wt. % of CMC and 4.83 wt. % of water.

This agglomerate was then exposed to fragrances. The resultant particles contained 20 wt. % of fragrances and 80 wt. % of the original agglomerate.

The particles still exhibited excellent powder and fragrance properties even after several weeks' storage at elevated temperatures (40° C.).

Comparison Composition B

The same zeolite-containing tower powder as from the first example was granulated with PEG 400. After drying, the resultant granular product comprised 94.98 wt. % of tower powder, 2.2 wt. % of PEG 400 and 2.82 wt. % of water.

This agglomerate was then exposed to fragrances. The resultant particles contained 20 wt. % of fragrances and 80 wt. % of the original agglomerate.

After production, the particles exhibited poor powder properties and were not flowable.

What is claimed:

1. A fluid-containing particle, comprising a carrier material agglomerated with a granulation liquid, the granulation liquid comprising a carbohydrate or carbohydrate derivative, and the particle further comprising an incorporated liquid.

2. The fluid-containing particle of claim 1, wherein the incorporated fluid comprises:
(a) an odoriferous substance;
(b) a liquid detergent or cleaning agent;
(c) a liquid cosmetic ingredient;
(d) a liquid non-pharmaceutical additive or active ingredient; or
(e) a mixture of the above.

3. The fluid-containing particle of claim 2, comprising at least 5% by weight of an odoriferous substance.

4. The fluid-containing particle of claim 1, wherein the carrier material comprises an anionic carrier material.

5. The fluid-containing particle of claim 4, wherein the anionic carrier material comprises zeolite or phyllosilicate.

6. The fluid-containing particle of claim 1, comprising at least 30% by weight of zeolite or bentonite.

7. The fluid-containing particle of claim 1, comprising at least 10% by weight of a solid material added in crystal form, based on the total solids content of the particle.

8. The fluid-containing particle of claim 1, wherein the carrier material comprises a spray-dried material.

9. The fluid-containing particle of claim 8, wherein the spray-dried material comprises a tower powder.

10. The fluid-containing particle of claim 1, wherein the starch or starch derivative comprises:
(a) a cellulose or cellulose derivative;
(b) a starch or starch derivative;
(c) a mono-, oligo- or polysaccharide or a derivative thereof; or
(d) a mixture of the above.

11. The fluid-containing particle of claim 1, wherein the granulation liquid comprises at least 80% by weight of a solvent, based on the granulation liquid.

12. The fluid-containing particle of claim 1, comprising a coating.

13. The particle of claim 12, wherein the coating comprises a powder or a film.

14. The fluid-containing particle of claim 12, wherein the coating comprises a colored substance, a dye, a brightener, or a pigment.

15. The fluid-containing particle of claim 12, comprising a thermoplastic coating material.

16. Fluid-containing particles according to claim 1, having an average particle size of 0.1 to 2.0 mm.

17. The fluid-containing particle of claim 1, comprising a solid silicate, phosphate, urea, urea derivative, sulfate, carbonate, citrate, citric acid, acetate, or salts of an anionic surfactant.

18. A detergent, cleaning agent, or conditioner comprising the fluid-containing particles of claim 1.

19. A method of fragrancing a room, vehicle, or closet, comprising contacting or exposing a room, vehicle, or closet in need of fragrancing with an effective amount of particles according to claim 1, wherein the particles comprise an odoriferous substance.

20. A method of fragrancing objects, comprising contacting or exposing an object in need of fragrancing with an effective amount of particles according to claim 1, wherein the particles comprise an odoriferous substance.
